(19) United States
(12) Patent Application Publication
(10) Pub. No.: US 2026/0028658 A1
(43) Pub. Date: Jan. 29, 2026

(54) LINEARIZED MRNA PREPARATION SYSTEM, USE THEREOF, AND PREPARATION METHOD FOR PREPARING MRNA BY USING SAME

(71) Applicants: CHONGQING PRECISION BIOTECH CO., LTD., Chongqing (CN); CHONGQING PRECISION BIOLOGICAL INDUSTRIAL TECHNOLOGY RESEARCH INSTITUTE CO., LTD., Chongqing (CN)

(72) Inventors: Hongtao Zhang, Chongqing (CN); Junjie Shen, Chongqing (CN); Yanmin Xu, Chongqing (CN); Yongchun Zhao, Chongqing (CN); Jiabing Ma, Chongqing (CN); Xiang Guo, Chongqing (CN); Xianxun Shi, Chongqing (CN); Maoxian Tian, Chongqing (CN)

(21) Appl. No.: 18/996,880

(22) PCT Filed: Jul. 21, 2023
(86) PCT No.: PCT/CN2023/108650

§ 371 Date: Jan. 17, 2025

(30) Foreign Application Priority Data
Jul. 29, 2022 (CN) 202210907876.9
Mar. 24, 2023 (CN) 202310301012.7

(51) Int. Cl. C12P 19/34; C12N 9/12; C12N 9/16

(52) U.S. Cl. CPC C12P 19/34; C12N 9/1247; C12N 9/16; C12Y 207/07006

(57) ABSTRACT

A linearized mRNA preparation system, use thereof, and a preparation method for preparing mRNA by using same. A linearization buffer system comprises: 0-150 mM Tris-Ac or phosphate, 0.5-150 mM divalent metal ions or trivalent metal ions, 0.1-0.75 U/μL restriction endonuclease, and 0-7.5 mM spermidine. The present invention provides a transcription system, which has higher translation efficiency and lower dsRNA generation compared with a conventional HEPES/Tris system, and a phosphate system can be used simultaneously with the novel All In One transcription system.

The present disclosure claims the priority to the Chinese patent application with the filing No. 202210907876.9 filed on Jul. 29, 2022 with the Chinese Patent Office, and entitled “Linearization Buffer System, RNA Preparation System, Preparation Method and Use”, the contents of which are incorporated herein by reference in entirety.

The present disclosure claims the priority to the Chinese patent application with the filing No. 202310301012.7 filed on Mar. 24, 2023 with the Chinese Patent Office, and entitled “Linearized mRNA Preparation System and Use thereof, and Preparation Method for Preparing mRNA by Using Same”, the contents of which are incorporated herein by reference in entirety.

### TECHNICAL FIELD

The present disclosure relates to the field of biotechnologies, and particularly to a linearized mRNA preparation system and thereof, and a preparation method for preparing mRNA by using the same.

### BACKGROUND ART

With the excellent performance of RNA vaccines in the COVID-19 pandemic, it is further demonstrated that medicines developed on the basis of RNA have great potential. As an emerging technology platform, RNA has advantages that DNA and protein do not have.

A Tris-HCl/HEPES-KOH buffer system is mostly used in a transcription stage for preparation of mRNA, in which HEPES is relatively costly and requires high-purity KOH to regulate pH; moreover, mRNA prepared by the conventional Tris-HCl system has a relatively high residual content of double-stranded RNA (dsRNA), thereby affecting downstream purification of mRNA, increasing difficulty in quality control and causing other problems. Double-stranded RNA (dsRNA) has been proved to be a major trigger factor of an immune pathway, and when synthesizing mRNA, for seeking minimization of cellular immune response during in vivo use, it is also crucial to eliminate these dsRNA contaminants from mRNA preparation or reduce their formation.

Therefore, there is an urgent need for a preparation system with a low cost and high purity, so as to solve problems such as purification and quality control.

### SUMMARY

In view of this, the present disclosure provides a linearized mRNA preparation system and use thereof, and a preparation method for preparing mRNA by using the same. The present disclosure, with use of a Tris-Ac or phosphate buffer, metal divalent ions or metal trivalent ions, can realize a new system, in which linearization, in vitro transcription, DNase I, capping and tailing are completed in sequence, without diluting or changing a new buffer system in midway. Compared with preparation methods in the prior art, this system has high translation efficiency, lower dsRNA generation, applicability to novel All In One transcription system and other characteristics.

In order to achieve the above objective of invention, the present disclosure provides following technical solutions.

In order to achieve the above objective of invention, the present disclosure provides following technical solutions.

The present disclosure provides a linearization buffer system, including:

| | | |
|---|---|---|
| Tris-Ac or phosphate | 0-150 | mM; |
| divalent metal ions or trivalent metal ions | 0.5-150 | mM; |
| restriction endonuclease | 0.1-0.75 | U/μL; |
| spermidine | 0-7.5 | mM. |

In some embodiments of the present disclosure, the above linearization buffer system includes:

| | | |
|---|---|---|
| Tris-Ac or phosphate | 0-120 | mM; |
| divalent metal ions or trivalent metal ions | 0.5-150 | mM; |
| restriction endonuclease | 0.1-0.75 | U/μL; |
| spermidine | 0-7.5 | mM. |

In some embodiments of the present disclosure, the above linearization buffer system includes:

| | | |
|---|---|---|
| Tris-Ac | 0-150 | mM; |
| divalent metal ions or trivalent metal ions | 0.5-150 | mM; |
| restriction endonuclease | 0.1-0.75 | U/μL; |
| spermidine | 0-7.5 | mM. |

In some embodiments of the present disclosure, the above linearization buffer system includes:

| | | |
|---|---|---|
| Tris-Ac | 0-120 | mM; |
| divalent metal ions or trivalent metal ions | 0.5-150 | mM; |
| restriction endonuclease | 0.1-0.75 | U/μL; |
| spermidine | 0-7.5 | mM. |

In some embodiments of the present disclosure, in the above linearization buffer system, the phosphate has a concentration of 15-75 mM.

In some embodiments of the present disclosure, in the above linearization buffer system, the phosphate is added in a form of HPO₄²⁻/H₂PO₄⁻.

In some embodiments of the present disclosure, in the above linearization buffer system, the HPO₄²⁻/H₂PO₄ is added in one or more forms of K₂HPO₄/KH₂PO₄, K₂HPO₄/NaH₂PO₄ or Na₂HPO₄/NaH₂PO₄.

In some embodiments of the present disclosure, in the above linearization buffer system, the phosphate has a pH value of 7.2-8.2.

In some embodiments of the present disclosure, in the above phosphate linearized system, the K₂HPO₄/KH₂PO₄ has a pH value of 7.2-8.0.

In some embodiments of the present disclosure, in the above phosphate linearized system, the K₂HPO₄/NaH₂PO₄ has a pH value of 7.2-8.2.

In some embodiments of the present disclosure, in the above phosphate linearized system, the Na₂HPO₄/NaH₂PO₄ has a pH value of 7.0-8.2.

In some embodiments of the present disclosure, in the above linearization buffer system, the divalent metal ions include one or more of Mg²⁺, Ca²⁺, Mn²⁺ or Zn²⁺; or the trivalent metal ions include Fe³⁺.

In some embodiments of the present disclosure, in the above linearization buffer system, the trivalent metal ions have a concentration of 25-150 mM; or
when the divalent metal ions are added in one or more forms of Mg²⁺, Mn²⁺ or Zn²⁺, the divalent metal ions have a concentration of 25-150 mM; or
when the divalent metal ions are added in the form of Ca²⁺, the divalent metal ions have a concentration of 0.5-1.5 mM.

In some embodiments of the present disclosure, in the above linearization buffer system, the Ca²⁺ is added in a form of CaAc₂ or CaCl₂; or
the Mg²⁺ is added in a form of MgAc₂ or MgCl₂; or
the Mn²⁺ is added in a form of MnCl₂ or MnAc₂; or
the Zn²⁺ is added in a form of ZnSO₄; or
the Fe³⁺ is added in a form of FeCl₃.

In some embodiments of the present disclosure, the above linearization buffer system includes:

| | | |
|---|---|---|
| Tris-Ac | 40 | mM; |
| MgAc₂ | 50 | mM; |
| Ca²⁺ | 0.5 | mM; |
| restriction endonuclease | 0.2 | U/μL; |
| spermidine | 2 | mM. |

In some embodiments of the present disclosure, the above linearization buffer system includes:

| | | |
|---|---|---|
| phosphate | 45 | mM; |
| MgAc₂ | 50 | mM; |
| Ca²⁺ | 0.5 | mM; |
| restriction endonuclease | 0.2 | U/μL; |
| spermidine | 2 | mM. |

In some embodiments of the present disclosure, the above linearization buffer system includes:

| | | |
|---|---|---|
| phosphate | 30 | mM; |
| MgAc₂ | 50 | mM; |
| Ca²⁺ | 0.5 | mM; |
| restriction endonuclease | 0.2 | U/μL; |
| spermidine | 2 | mM. |

In some embodiments of the present disclosure, the above linearization buffer system includes:

| | |
|---|---|
| Tris-Ac | 0 mM, 30 mM, 40 mM, 50 mM, 80 mM, 120 mM or 150 |
| | mM; |
| MgAc₂ | 0 mM, 25 mM, 40 mM, 50 mM, 60 mM, 100 mM or 150 |
| | mM; |
| Ca²⁺ | 0 mM, 0.25 mM, 0.5 mM, 1 mM or 1.5 mM; |
| restriction | 0 U/μL, 0.1 U/μL, 0.2 U/μL, 0.4 U/μL, 0.6 U/μL or 0.75 |
| endonuclease | U/μL; |
| spermidine | 0 mM, 1 mM, 2 mM, 4 mM, 6 mM or 7.5 mM. |

In some embodiments of the present disclosure, the above linearization buffer system includes:

| | |
|---|---|
| phosphate | 30 mM or 45 mM; |
| MgAc₂ | 0 mM, 25 mM, 40 mM, 50 mM, 60 mM, 100 mM or 150 |
| | mM; |
| Ca²⁺ | 0 mM, 0.25 mM, 0.5 mM, 1 mM or 1.5 mM; |
| restriction | 0 U/μL, 0.1 U/μL, 0.2 U/μL, 0.4 U/μL, 0.6 U/μL or 0.75 |
| endonuclease | U/μL; |
| spermidine | 0 mM, 1 mM, 2 mM, 4 mM, 6 mM or 7.5 mM. |

In some embodiments of the present disclosure, the above linearization buffer system further includes:

| | |
|---|---|
| template | 0 μg/μL, 0.025 μg/μL, 0.05 μg/μL, 0.075 μg/μL, 0.1 μg/μL, |
| | 0.15 μg/μL or 0.2 μg/μL. |

The present disclosure further provides use of the above linearization buffer system in cotranscription system.

The present disclosure further provides use of the above linearization buffer system in preparation of RNA by in vitro transcription.

The present disclosure further provides a cotranscription system, including the above linearization buffer system and an acceptable enzyme or other adjuvants.

In some embodiments of the present disclosure, the template in the above linearization buffer system includes one or more of R5, R39, R53, H18 or pSFV-12.

In some embodiments of the present disclosure, in the template in the above linearization buffer system, the R5 or the R39 is SARS-CoV-2 Spike WT (6028 bp); the R53 is SARS-CoV-2 Spike Omicron (5951 bp); the H18 is HPV (2481 bp); and the pSFV-12 is SFV saRNA (11413 bp).

In some embodiments of the present disclosure, in the above linearization buffer system, the Tris-Ac can be replaced with one or more of HEPES and KOH, Tris-MES, Tris-HCl, K₂HPO₄ and KH₂PO₄, K₂HPO₄ and NaH₂PO₄ or MOPS-Ac.

In some embodiments of the present disclosure, in the above linearization buffer system, the MgAc₂ can be replaced with one or more of MnCl₂, MnAc₂, ZnSO₄, CaAc₂, CaCl₂, MgCl₂ or FeCl₃.

In some embodiments of the present disclosure, in the above linearization buffer system, the CaCl₂ can be replaced with one or more of MnCl₂, MnAc₂, ZnSO₄, MgCl₂, NaAc, NaCl, KCl, KAc or FeCl₃.

In some embodiments of the present disclosure, in the above linearization buffer system, the restriction endonuclease includes one or more of EcoRI, BamHI, BspQI or BsaI.

The present disclosure further provides a preparation system of RNA, including the above linearization buffer system and an acceptable enzyme or other adjuvants.

In some embodiments of the present disclosure, the above preparation system further includes: a transcription system, where
the transcription system includes:

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM; |
| CAP | 2-18 | mM; |

or

| | | |
|---|---|---|
| T7 polymerase | 1.25-10 | U/μL; |
| IPP | 0-0.015 | U/μL; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM; |
| CAP | 2-18 | mM; |

or

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM. |

In some embodiments of the present disclosure, the transcription system in the above preparation system includes:

| | | |
|---|---|---|
| T7 polymerase | 2.5-10 | U/μL; |
| IPP | 0.0025-0.015 | U/μL; |
| DTT | 0-75 | mM; |
| RNase inhibitor | 0-5 | U/μL; |
| rATP | 7-16 | mM; |
| rCTP | 7-16 | mM; |
| rUTP | 7-16 | mM; |
| rGTP | 7-16 | mM; |
| CAP | 7-16 | mM; |

or

| | | |
|---|---|---|
| T7 polymerase | 2.5-10 | U/μL; |
| IPP | 0.0025-0.015 | U/μL; |
| RNase inhibitor | 0-5 | U/μL; |
| rATP | 7-16 | mM; |
| rCTP | 7-16 | mM; |
| rUTP | 7-16 | mM; |
| rGTP | 7-16 | mM; |

or

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM. |

In some embodiments of the present disclosure, the transcription system in the above preparation system includes:

| | |
|---|---|
| T7 | 0 U/μL, 1.25 U/μL, 2.5 U/μL, 5 U/μL, 7.5 U/μL or 10 |
| polymerase | U/μL; |
| IPP | 0 U/μL, 0.0025 U/μL, 0.00625 U/μL, 0.00875 U/μL, |
| | 0.0125 U/μL or 0.015 U/μL; |
| DTT | 0 mM, 10 mM, 20 mM, 25 mM, 50 mM or 75 mM; |
| RNase | 0 U/μL, 1 U/μL, 2 U/μL, 4 U/μL or 5 U/μL; |
| inhibitor | |
| rATP | 2 mM, 5 mM, 7 mM, 7.5 mM, 14 mM, 16 mM or 18 mM; |
| rCTP | 2 mM, 5 mM, 7 mM, 7.5 mM, 14 mM, 16 mM or 18 mM; |
| rUTP | 2 mM, 5 mM, 7 mM, 7.5 mM, 14 mM, 16 mM or 18 mM; |
| rGTP | 2 mM, 5 mM, 7 mM, 7.5 mM, 14 mM, 16 mM or 18 mM; |
| CAP | 7.5 mM or 14 mM. |

In some embodiments of the present disclosure, the transcription system in the above preparation system includes:

| | | |
|---|---|---|
| T7 polymerase | 5 | U/μL; |
| IPP | 0.00625 | U/μL; |
| DTT | 20 | mM; |
| RNase inhibitor | 1 | U/μL; |
| rATP | 14 | mM; |
| rCTP | 14 | mM; |
| rUTP | 14 | mM; |
| rGTP | 14 | mM; |
| CAP | 14 | mM. |

In some embodiments of the present disclosure, the CAP in the above preparation system can be m7G(5′)ppp(5′)G, m7G(3oMe)(5′)ppp(5′)G, m7G(5′)ppp(5′)ApG, m7G(3oMe)(5′)ppp(5′)(A)pG, m7G(5′)ppp(5′)(2oMeA)pG, m7G(3oMe)(5′)ppp(5′)(2oMeA)pG or m7G(3oMe)(5′)ppp(5′)(m6A)pG and corresponding modified cap structure analogues, and for different cap structure analogues, only corresponding replacement of transcription initiation site of T7 promoter on the template is needed; therefore, CAP can be any one of the above.

In some embodiments of the present disclosure, the CAP in the above preparation system is clean CAP AG.

In some embodiments of the present disclosure, in the above preparation system, the preparation system includes:

| | | |
|---|---|---|
| Tris-Ac | 40 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| restriction endonuclease | 0.2 | U/μL; |
| T7 polymerase | 5 | U/μL; |
| IPP | 0.00625 | U/μL; |
| DTT | 20 | mM; |
| RNase inhibitor | 1 | U/μL; |

or

| | | |
|---|---|---|
| Tris-Ac | 40 | mM; |
| rATP | 14 | mM; |
| rCTP | 14 | mM; |
| rUTP | 14 | mM; |
| rGTP | 14 | mM; |
| MgAc₂ | 50 | mM; |
| CaCl₂ | 0.5 | mM; |
| spermidine | 2.0 | mM; |
| restriction endonuclease | 0.2 | U/μL; |
| T7 polymerase | 5 | U/μL; |
| IPP | 0.00625 | U/μL; |
| DTT | 20 | mM; |
| RNase inhibitor | 1 | U/μL; |

or

| | | |
|---|---|---|
| Tris-Ac | 40 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| restriction endonuclease | 0.2 | U/μL; |
| T7 polymerase | 5 | U/μL; |
| IPP | 0.00625 | U/μL; |
| RNase inhibitor | 1 | U/μL; |

or

| | | |
|---|---|---|
| K₂HPO₄/KH₂PO₄ | 45 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| T7 polymerase | 5 | U/μL; |
| IPP | 0.0075 | U/μL; |
| DTT | 25 | mM; |
| RNase inhibitor | 1 | U/μL; |

or

| | | |
|---|---|---|
| K₂HPO₄/KH₂PO₄ | 30 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| T7 polymerase | 5 | U/μL; |
| IPP | 0.0075 | U/μL; |
| DTT | 25 | mM; |
| RNase inhibitor | 1 | U/μL. |

The present disclosure further provides a preparation method for RNA, including: mixing the above preparation system with the template, where preferably, the template is plasmid.

In some embodiments of the present disclosure, the template in the above preparation method has a concentration of 0-7.5 mM.

In some embodiments of the present disclosure, the template in the above preparation method has a concentration of 2 mM.

In some embodiments of the present disclosure, the mixing in the above preparation method includes following steps:
S1: mixing the template with the linearization buffer system, to render a sample; and
S2: mixing the sample obtained in S1 with the transcription system in the above preparation system.

In some embodiments of the present disclosure, the above preparation method further includes, after the mixing, steps of removing DNA and centrifuging to remove supernatant.

In some embodiments of the present disclosure, the mixing in S1 in the above preparation method is performed at a temperature of 50° C. for a period of 1 h.

In some embodiments of the present disclosure, the mixing in S2 in the above preparation method is performed at a temperature of 37° C. for a period of 3 h.

The present disclosure further provides use of the above linearization buffer system, the above cotranscription system or the above preparation system in preparation of RNA.

The present disclosure further provides a RNA preparation kit, including the above linearization buffer system, the above cotranscription system or the above preparation system and an acceptable adjuvant or vector.

The linearization buffer system includes:

| | | |
|---|---|---|
| Tris-Ac or phosphate | 0-150 | mM; |
| divalent metal ions or trivalent metal ions | 0.5-150 | mM; |
| restriction endonuclease | 0.1-0.75 | U/μL; |
| spermidine | 0-7.5 | mM. |

The present disclosure has the following beneficial effects.
(1) The present disclosure provides a restriction endonuclease system with multiple definite components, without animal-derived components; and Tris-Ac in the linearization buffer system can be replaced with HEPES/KOH, Tris-MES, Tris-HCl, K₂HPO₄/KH₂PO₄, K₂HPO₄/NaH₂PO₄, and MOPS-Ac, but the use of Tris-Ac has higher yield and is more stable, and all of the above systems are conventional neutral buffer systems in laboratories and are convenient to use.
(2) The present disclosure provides a plurality of high-yield systems for preparing mRNA by in vitro transcription, which can be used for cotranscription or non-cotranscription systems, and unit yield reaches 17 mg/mL.
(3) The present disclosure provides an efficient template digestion system, and addition of Mg²⁺/Ca²⁺ can promote activity of different DNase I to degrade DNA templates with different sizes to fragments smaller than 50 bp, thus being beneficial to further reducing exogenous DNA residues in mRNA stock solution, and meanwhile reducing pressure of a downstream purification process.
(4) The system provided in the present disclosure can realize a continuous preparation process from a plasmid template to mRNA, without additional liquid change and purification operation, realizes high efficiency and saves time, manpower and process cost in process manufacturing.
(5) The system provided in the present disclosure has high applicability, and can be used for preparing different templates.
(6) The present disclosure provides a plurality of phosphate-based in vitro transcription systems, which can be used for preparing mRNA, with unit yield reaching 15 mg/mL; moreover, the phosphate system can be any one of K₂HPO₄/KH₂PO₄, K₂HPO₄/NaH₂PO₄, Na₂HPO₄/NaH₂PO₄, etc.
(7) The present disclosure provides a transcription system that has higher translation efficiency than conventional HEPES/Tris system.
(8) The present disclosure provides a transcription system that has lower dsRNA generation than the conventional HEPES/Tris system.
(9) The phosphate system in the present disclosure can be applied to the novel All In One transcription system.

### BRIEF DESCRIPTION OF DRAWINGS

FIG. 1 shows analysis of linearization under different conditions, where sequentially from left to right: R-N: template is plasmid (NTP is not added in a linearization stage); R-D: template is plasmid (DTT is not added in the linearization stage); R-ND: template is plasmid (DTT and NTP are not added in the linearization stage); S-L: linearized DNA control; R5: plasmid DNA control;

FIG. 2 shows analysis of purity of mRNAs under different conditions;

FIG. 3 shows analysis of yield of mRNAs under different conditions;

FIG. 4 shows analysis of mRNAs prepared by two processes by non-denaturing agarose gel electrophoresis analysis;

FIG. 5 shows statistics of yield of mRNAs prepared by two processes;

FIG. 6 shows expression of mRNAs prepared by two processes after being electrotransferred to 293T cells by ELISA assay;

FIG. 7 shows orthogonal screening for mRNA high-yield system with Tris-Ac/Tris-HCl system;

FIG. 8 shows statistics of yield of mRNA high-yield system obtained by orthogonal screening with Tris-Ac/Tris-HCl system, where the left shows Tirs-Ac, and the right shows Tris-HCl, where unit is UL; DTT 0.5 M; magnesium acetate 1 M; Tris-HCl/Ac 1 M, where K1 shows sums of yields at a first concentration level of a single factor; K2 shows sums of yields at a second concentration level of a single factor; K3 shows sums of yields at a third concentration level of a single factor; k1 shows mean yields at the first concentration level of a single factor; k2 shows mean yields at the second concentration level of a single factor; k3 shows mean yields at the third concentration level of a single factor; and R shows ranges of k values of different concentrations of different factors;

FIG. 9 shows analysis of purity of mRNA prepared by the Tris-Ac/Tris-HCl system (OligodT column chromatography purification), where sequentially from left to right: M; Tirs-Ac (IVT); Tris-Ac (Flu); Tris-Ac (Elu); Tris-HCl (IVT); Tris-HCl (Flu); Tris-HCl (Elu), where IVT (refers to purity of RNA at the end of in vitro transcription); Flu (refers to purity of sample in OligdT flow-through fraction of purification); Elu (refers to purity of sample in OligdT eluate of purification);

FIG. 10 shows purity of mRNA prepared by Tris-Ac/Tris-HCl orthogonal system by HPLC analysis, where an upper drawing shows Tris-HCl, and a lower drawing shows Tris-Ac;

FIG. 11 shows expression of mRNA prepared by the Tris-Ac/Tris-HCl orthogonal system in 293T cells by ELISA assay, where LiCl+EtOH: lithium chloride precipitation method; OligodT: chromatography purification;

FIG. 12 shows analysis of influence of different Tris-Ac/MgAc₂/CaCl₂ concentrations on linearization, where amounts added in the drawing are corresponding to final concentrations in TABLE 11;

FIG. 13 shows analysis of influence of different Tris-Ac/MgAc₂/CaCl₂ concentrations on DNase I activity, where detection conditions are 37° C./60 min, and amounts added in the drawing are corresponding to final concentrations in TABLE 12;

FIG. 14 shows analysis of influence of different Tris-Ac/MgAc₂/CaCl₂ concentrations on yield of in vitro transcribed mRNA;

FIG. 15 shows analysis of different Tris-Ac/MgAc₂/CaCl₂ concentrations on purity of in vitro transcribed mRNA, where amounts added in the drawing are corresponding to final concentrations in TABLE 11; a left drawing shows influence of different amounts of Tris-AC added on the purity of mRNA; and a right drawing shows influence of different amounts of magnesium ions and calcium ions added on the purity of mRNA;

FIG. 16 shows analysis of activity of DNase I from different manufacturers in different CaCl₂ concentrations, where detection conditions are 37° C./30 min;

FIG. 17 shows analysis of activity of DNase I from different manufacturers in different CaCl₂ concentrations, where detection conditions are 37° C./60 min;

FIG. 18 shows analysis of influence of different spermidine concentrations on linearization/DNase I, where amounts added in the drawing are corresponding to final concentrations in TABLE 14;

FIG. 19 shows analysis of influence of different spermidine concentrations on yield of in vitro transcribed mRNA, where amounts added in the drawing are corresponding to final concentrations in TABLE 14;

FIG. 20 shows analysis of different spermidine concentrations on purity of in vitro transcribed mRNA, where amounts added in the drawing are corresponding to final concentrations in TABLE 14;

FIG. 21 shows analysis of different IPP/DTT/RNase-Inhibitor/T7 enzyme concentrations on purity of in vitro transcribed mRNA, where amounts added in the drawing are corresponding to final concentrations in TABLE 16;

FIG. 22 shows analysis of influence of different RNase-Inhibitor enzyme concentrations on yield of in vitro transcribed mRNA, where amounts added in the drawing are corresponding to final concentrations in TABLE 16;

FIG. 23 shows analysis of different T7 enzyme concentrations on purity of in vitro transcribed mRNA, where amounts added in the drawing are corresponding to final concentrations in TABLE 16;

FIG. 24 shows analysis of different IPP concentrations on purity of in vitro transcribed mRNA, where amounts added in the drawing are corresponding to final concentrations in TABLE 16;

FIG. 25 shows analysis of different DTT concentrations on purity of in vitro transcribed mRNA, where amounts added in the drawing are corresponding to final concentrations in TABLE 16;

FIG. 26 shows analysis of influence of different BspQI/template R53/NTP (CleanCap) concentrations on linearization, where amounts added in the drawing are corresponding to final concentrations in TABLE 21;

FIG. 27 shows analysis of influence of different BspQI/template R53/NTP (CleanCap) concentrations on DNase I, where detection conditions are 37° C./30 min, and amounts added in the drawing are corresponding to final concentrations in TABLE 21;

FIG. 28 shows analysis of influence of different BspQI/template R53/NTP (CleanCap) concentrations on DNase I, where detection conditions are 37° C./60 min, and amounts added in the drawing are corresponding to final concentrations in TABLE 21;

FIG. 29 shows analysis of influence of different BspQI/template R53/NTP (CleanCap) concentrations on purity of mRNA, where amounts added in the drawing are corresponding to final concentrations in TABLE 21;

FIG. 30 shows analysis of influence of different BspQI concentrations on yield of mRNA, where amounts added in the drawing are corresponding to final concentrations in TABLE 21;

FIG. 31 shows analysis of influence of different template R53 concentrations on yield of mRNA;

FIG. 32 shows analysis of influence of different NTP (CleanCap) concentrations on yield of mRNA;

FIG. 33 shows analysis of linearization of different templates in All In One system, where SapI represents that the template is linearized by this enzyme in the All In One system; plasmid represents negative control; R5/R39: SARS-CoV-2 Spike WT (6028 bp); R53: SARS-CoV-2 Spike Omicron (5951 bp); H18: HPV (2481 bp); pSFV-12: SFV saRNA (11413 bp);

FIG. 34 shows DNase I analysis after linearization of different templates in the All In One system;

FIG. 35 shows linearization of R5 plasmid with different restriction enzymes in All In One system and DNase I analysis

FIG. 36 shows influence of different metal ions replacing MgAc₂ on linearization;

FIG. 37 shows influence of different metal ions replacing MgAc₂ on DNase I activity, where detection conditions are 37° C./30 min;

FIG. 38 shows influence of different metal ions replacing MgAc₂ on DNase I activity, where detection conditions are 37° C./60 min;

FIG. 39 shows influence of different metal ions replacing MgAc₂ on purity of in vitro transcribed mRNA;

FIG. 40 shows influence of different metal ions replacing MgAc₂ on yield of in vitro transcribed mRNA;

FIG. 41 shows influence of different metal ions replacing CaCl₂) on linearization;

FIG. 42 shows influence of different metal ions replacing CaCl₂ on DNase I, where detection conditions are 37° C./30 min;

FIG. 43 shows influence of different metal ions replacing CaCl₂ on DNase I, where detection conditions are 37° C./60 min;

FIG. 44 shows influence of different metal ions replacing CaCl₂ on yield of mRNA;

FIG. 45 shows influence of different metal ions replacing CaCl₂ on purity of mRNA;

FIG. 46 shows influence of different buffers replacing Tris-HCl on linearization, where sequentially from left to right: 1 #HEPES/KOH; 2 #Tris-MES; 3 #Tris-HCl; 4 #Tris-Ac; 5 #K₂HPO₄/KH₂PO₄; 6 #K₂HPO₄/NaH₂PO₄; 7 #MOPS-Ac; all being 1M (Ph 8.0);

FIG. 47 shows influence of different buffers replacing Tris-HCl on DNase I activity, where sequentially from left to right: 1 #HEPES/KOH; 2 #Tris-MES; 3 #Tris-HCl; 4 #Tris-Ac; 5 #K₂HPO₄/KH₂PO₄; 6 #K₂HPO₄/NaH₂PO₄; 7 #MOPS-Ac; all being 1 M (Ph8.0);

FIG. 48 shows influence of different buffers replacing Tris-HCl on yield of mRNA, where sequentially from left to right: 1 #HEPES/KOH; 2 #Tris-MES; 3 #Tris-HCl; 4 #Tris-Ac; 5 #K₂HPO₄/KH₂PO₄; 6 #K₂HPO₄/NaH₂PO₄; 7 #MOPS-Ac; all being 1 M (Ph 8.0);

FIG. 49 shows influence of different buffers replacing Tris-HCl on purity of mRNA, where sequentially from left to right: 1 #HEPES/KOH; 2 #Tris-MES; 3 #Tris-HCl; 4 #Tris-Ac; 5 #K₂HPO₄/KH₂PO₄; 6 #K₂HPO₄/NaH₂PO₄; 7 #MOPS-Ac; all being 1 M (Ph 8.0);

FIG. 50 shows comparison of translation efficiency of RNAs prepared by different systems;

FIG. 51 shows comparison of translation efficiency of RNAs prepared by K₂HPO₄/NaH₂PO₄ with different pH;

FIG. 52 shows comparison of translation efficiency of RNAs prepared by K₂HPO₄/KH₂PO₄ with different pH;

FIG. 53 shows comparison of translation efficiency of RNAs prepared by Na₂HPO₄/NaH₂PO₄ with different pH;

FIG. 54 shows comparison of purity of RNA stock solutions prepared by different systems;

FIG. 55 shows analysis of dsRNA impurity of RNA stock solutions prepared by different systems;

FIG. 56 shows comparison of concentrations of RNAs prepared by different systems;

FIG. 57 shows statistics of different amounts of T7 enzyme and reaction duration on RNA concentrations;

FIG. 58 shows analysis of purity of RNAs prepared by different amounts of T7 enzyme and reaction duration;

FIG. 59 shows analysis of correlation between translation efficiency and time of RNAs prepared by different amounts of T7 enzyme and reaction duration;

FIG. 60 shows influence of different amounts of DTT added on linearization of different plasmids and template digestion;

FIG. 61 shows analysis of purity of RNAs from phosphate system All In One and cotranscription, where from left to right, DNA Marker, R53 plasmid linearization, pSFV-12 plasmid linearization, R53 template digestion and pSFV-12 template digestion are respectively shown, volumes of 0.5 M DTT used are 0 μL, 0.25 μL, 0.5 μL and 1.0 μL.

### DETAILED DESCRIPTION OF EMBODIMENTS

The present disclosure discloses a linearized mRNA preparation system and a use thereof, and a preparation method for preparing mRNA by using the same. Those skilled in the art could appropriately improve process parameter for implementation with reference to contents herein. It should be particularly indicated that all similar replacements and modifications would be obvious to those skilled in the art, all of which are considered to be included in the present disclosure. The method and use of the present disclosure are described with preferred embodiments, and it would be apparent to those concerned that modifications or appropriate variations and combinations of the method and use described herein can be made to implement and use the technology in the present disclosure, without departing from the contents, spirit, and scope of the present disclosure.

Technical solutions provided in the present disclosure include the following.

In the first aspect, the present disclosure provides a universal system for preparing RNA by in vitro transcription, and components and amounts in the system, including ranges and optimal values;

| | |
|---|---|
| Tris-Ac(pH 8.0; 1M) | 0 mM~150 mM |
| rATP(200 mM) | 2 mM~18 mM |
| rCTP(200 mM) | 2 mM~18 mM |
| rUTP(200 mM) | 2 mM~18 mM |
| rGTP(200 mM) | 2 mM~18 mM |
| CleanCap AG(100 mM) | 2 mM~18 mM |
| Plasmid | 0.025 μg/μL~0.2 μg/μL |
| MgAc₂(1M) | 25 mM~150 mM |
| CaCl₂(10 mM) | 0.5 mM~1.5 mM |
| Spermidine (100 mM) | 0 mM~7.5 mM |
| BspQI(10000 U/mL) | 0.1 U/μL~0.75 U/μL |
| RNase free Water | up to 20 μL |
| IPP(100 U/mL) | 0 U/μL~0.015 U/μL |
| DTT(500 mM) | 0 mM~75 mM |
| RNase Inhibitor(40000 U/mL) | 0 U/μL~5 U/μL |
| T7 polymerase(50000 U/ml) | 1.25 U/μL~10 U/μL; |

or

| | |
|---|---|
| Tris-Ac(pH 8.0; 1M) | 0 mM~120 mM |
| rATP(200 mM) | 7 mM~16 mM |
| rCTP(200 mM) | 7 mM~16 mM |
| rUTP(200 mM) | 7 mM~16 mM |
| rGTP(200 mM) | 7 mM~16 mM |
| CleanCap AG(100 mM) | 7 mM~16 mM |
| Plasmid | 0.05 μg/μL~0.2 μg/μL |
| MgAc₂(1M) | 50 mM~100 mM |
| CaCl₂(10 mM) | 0.5 mM~1.5 mM |
| Spermidine (100 mM) | 0 mM~7.5 mM |
| BspQI(10000 U/mL) | 0.1 U/μL~0.75 U/μL |
| RNase free Water | up to 20 μL |
| IPP(100 U/mL) | 0.0025 U/μL~0.015 U/μL |
| DTT(500 mM) | 0 mM~75 mM |
| RNase Inhibitor(40000 U/mL) | 0 U/μL~5 U/μL |
| T7 polymerase(50000 U/ml) | 2.5 U/μL~10 U/μL; |

or

| Tris-Ac(pH 8.0; 1M) | 40 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Plasmid | 0.075 | μg/μL |
| MgAc₂(1M) | 50 | mM |
| CaCl₂(10 mM) | 0.5 | mM |
| Spermidine (100 mM) | 2.0 | mM |
| BspQI(10000 U/mL) | 0.2 | U/μL |
| RNase free Water | up to 20 μL | |
| IPP(100 U/mL) | 0.00625 | U/μL |
| DTT(500 mM) | 20 | mM |
| RNase Inhibitor(40000 U/mL) | 1 | U/μL |
| T7 polymerase(50000 U/ml) | 5 | U/μL |

In the second aspect, the present disclosure further provides a process for preparing RNA based on the above system.

Preparation process includes: thawing components of the linearization buffer system to a room temperature, mixing Tris-Ac, magnesium acetate, spermidine, calcium chloride, purified plasmid, BspQI, NTP, CleanCap (Trilink) and water uniformly, placing mixture in a PCR instrument or a metal bath to react at 50° C. for 1 h, at the end of reaction, adding corresponding amounts of T7, DTT, IPP and RNase-Inhibitor and mixing uniformly, to render a final solution with a total volume of at most 20 μL, further reacting in the PCR instrument or metal bath at 37° C. for 3 h, at the end of reaction, adding 3 μL of DNase I, holding at 37° C. for 30 min (37° C./30 min), further adding 10 μL of lithium chloride precipitation solution (Thermofisher), mixing uniformly, holding at −20° C. for 30 min, centrifuging at 4° C. at 15,000 RPM for 15 min to remove supernatant, washing twice with 1 mL of 75% ethanol, centrifuging again at 4° C. at 15,000 RPM for 15 min to remove supernatant, and re-suspending RNA precipitate with water, TE, etc. so as to render mRNA including a cap and a tail.

In the third aspect, the present disclosure further provides a phosphate universal system for preparing RNA by in vitro transcription, and components and amounts in the system, including ranges and optimal values;

| phosphate | 15~75 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.0675 | μg/μL |
| MgAc₂(1M) | 50 | mM |
| CaCl₂(10 mM) | 0.5 | mM |
| Spermidine (100 mM) | 2.0 | mM |
| RNase free Water | up to 20 μL | |
| IPP(100 U/mL) | 0.0075 | U/μL |
| DTT(500 mM) | 0~25 | mM |
| RNase Inhibitor(40000 U/mL) | 1 | U/μL |
| T7 polymerase(50000 U/mL) | 1.25~7.5 | U/μL; |

or

| phosphate | 45 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.0675 | μg/μL |
| MgAc₂(1M) | 50 | mM |
| CaCl₂(10 mM) | 0.5 | mM |
| Spermidine (100 mM) | 2.0 | mM |
| RNase free Water | up to 20 μL | |
| IPP(100 U/mL) | 0.0075 | U/μL |
| DTT(500 mM) | 25 | mM |
| RNase Inhibitor(40000 U/mL) | 1 | U/μL |
| T7 polymerase(50000 U/mL) | 5 | U/μL; |

or

| phosphate | 30 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.0675 | μg/μL |
| MgAc₂(1M) | 50 | mM |
| CaCl₂(10 mM) | 0.5 | mM |
| Spermidine (100 mM) | 2.0 | mM |
| RNase free Water | up to 20 μL | |
| IPP(100 U/mL) | 0.0075 | U/μL |
| DTT(500 mM) | 25 | mM |
| RNase Inhibitor(40000 U/mL) | 1 | U/μL |
| T7 polymerase(50000 U/mL) | 5 | U/μL. |

In the above, the phosphate buffer system can be replaced with common phosphate buffers such as K₂HPO₄/KH₂PO₄, K₂HPO₄/NaH₂PO₄ and Na₂HPO₄/NaH₂PO₄.

In the fourth aspect, the present disclosure further provides a process for preparing RNA by a phosphate universal system. Specific steps are as follows: a preparation process: mixing phosphate, magnesium acetate, spermidine, calcium chloride, a linear template, BspQI, NTP, a cap analogue (CleanCap AG (m7(3′OMeG)(5′)ppp(5′)(2′OMeA)pG)), T7, DTT, IPP, RNase-Inhibitor and water uniformly, to render a final solution with a total volume of at most 20 μL, reacting at 37° C. for 3 h, at the end of reaction, adding 3 μL of DNase I at 37° C. for 30 min, adding 10 ul of lithium chloride precipitation solution (Thermofisher), mixing uniformly, holding at −20° C. for 30 min, centrifuging at 4° C. at 15,000 RPM for 15 min to remove supernatant, washing twice with 1 mL of 75% ethanol, centrifuging again at 4° C. at 15,000 RPM for 15 min to remove supernatant, and re-suspending RNA precipitate with water, TE, etc. to render mRNA including a cap and a tail.

A lithium chloride precipitation method in the present disclosure includes the following steps:
1) at the end of reaction, adding 10 μL of lithium chloride precipitation solution and mixing uniformly, and placing at −20° C. for 30 min;
2) centrifuging in a centrifuge at 4° C. at 15,000 RPM for 15 min, to remove supernatant, and washing with 70% ethanol twice; and
3) centrifuging in the centrifuge at 4° C. at 15,000 RPM for 15 min, to remove supernatant, drying in a fume hood, re-suspending by adding RNase free water, and measuring concentration by a Nanodrop.

In a screening process of All In One system of the present disclosure and characterization of concentration ranges of components of the All In One system as well as component replacement, raw materials and reagents used are all commercially available.

The present disclosure will be further illustrated below in conjunction with examples.

### Example 1 All In One Screening Process

1. All In One technology optimization—time point of adding NTP/DTT and endonuclease inactivation pair (NTP 7.5 mM)

### Experimental Steps

System configurations A-C are corresponding to TABLE 1 to TABLE 3 in turn.

TABLE 1
Addition of DTT and NTP/CleanCap in Linearization Stage

| | | |
|---|---|---|
| Tris-Ac(pH 8.0; 1M) | 30 | mM |
| rATP(200 mM) | 7.5 | mM |
| rCTP(200 mM) | 7.5 | mM |
| rUTP(200 mM) | 7.5 | mM |
| rGTP(200 mM) | 7.5 | mM |
| CleanCap AG(100 mM) | 7.5 | mM |
| Template | 0.075 | μg/μL |
| MgAc₂(1M) | 40 | mM |
| CaCl₂(10 mM) | 0.5 | mM |
| Spermidine (100 mM) | 2.0 | mM |
| BspQI(10000 U/mL) | 0.2 | U/μL |
| DTT(500 mM) | 20 | mM |

| | |
|---|---|
| RNase free Water | to 20 μL |

TABLE 2
Addition of DTT in Linearization Stage

| | | |
|---|---|---|
| Tris-Ac(pH 8.0; 1M) | 30 | mM |
| Template | 0.075 | μg/μL |
| MgAc₂(1M) | 40 | mM |
| CaCl₂(10 mM) | 0.5 | mM |
| Spermidine (100 mM) | 2.0 | mM |
| BspQI(10000 U/mL) | 0.2 | U/μL |
| DTT(500 mM) | 20 | mM |

| | |
|---|---|
| RNase free Water | to 20 μL |

TABLE 3
Addition of NTP/CleanCap in Linearization Stage

| | | |
|---|---|---|
| Tris-Ac(pH 8.0; 1M) | 30 | mM |
| rATP(200 mM) | 7.5 | mM |
| rCTP(200 mM) | 7.5 | mM |
| rUTP(200 mM) | 7.5 | mM |
| rGTP(200 mM) | 7.5 | mM |
| CleanCap AG(100 mM) | 7.5 | mM |
| Template | 0.075 | μg/μL |
| MgAc₂(1M) | 40 | mM |
| CaCl₂(10 mM) | 0.5 | mM |
| Spermidine (100 mM) | 2.0 | mM |
| BspQI(10000 U/mL) | 0.2 | U/μL |

| | |
|---|---|
| RNase free Water | to 20 μL |

All of the above used two templates: a R5 (SARS-CoV-2 Spike WT; 6028 bp) plasmid DNA template and a pre-linearized DNA template, where each template was configured with two replicates, and RNase free water was finally added to render a final in vitro transcription system 20 μL (IPP/T7/RNase-In volume was removed in the linearization stage). R5 construction method: assembling T7 promoter (transcription initiation site being GGG), R globin 5′UTR/3′UTR, SARS-Cov-2 Spike (NCBI Reference Sequence: NC_045512.2) and polyA together by a molecular biological method and finally ligating with a pUC57 cloning vector to form R5.
1) The system was mixed uniformly and placed at 50° C. for 1 h, and at the end of reaction, 1 μL was taken to measure linearization efficiency; an enzyme inactivation group was subjected to inactivation of restriction endonuclease at 80° C. for 20 min, and the other group was not inactivated; afterwards, IPP (100 U/mL) 0.8 μL, RNase Inhibitor (40,000 U/mL) 0.5 μL, and T7 polymerase (50,000 U/mL) 2 μL were added; in addition, groups without addition of DTTh and/or NTP (CleanCap) in the linearization stage were additionally added with corresponding amounts of the above reagents respectively and mixed uniformly;
2) resultant was placed at 37° C. for 3 h, added with 3 μL of DNase I at the end of reaction and mixed uniformly, and placed at 37° C. for 30 min;
3) at the end of reaction, purification was performed by a lithium chloride precipitation method; and
4) 1.5 μL of RNA sample was additionally taken, mixed with 9 μL of RNase free water and 2 μL of 6× loading buffer uniformly, and analyzed for purity/integrity/size by 1% non-denaturing agarose gel electrophoresis.

Yield of mRNA under different conditions was analyzed. DTT-free experimental group in the linearization stage of the plasmid group was named as R-DTT, NTP-free experimental group in the linearization stage of the plasmid group was named as R-NTP, and DTT&NTP-free experimental group in the linearization stage of the plasmid group was named as R-D&N. DTT-free experimental group in THE linearization stage of the pre-linearized template was named as L-DTT, NTP-free experimental group in the linearization stage of the pre-linearized template was named as L-NTP, and DTT&NTP-free experimental group in the linearization stage of the pre-linearized template was named as L-D&N.

TABLE 4
Analysis of Yield of mRNA under Different Conditions

80° C.

| Name of experimental group | 20 min | 0 min |
|---|---|---|
| R-DTT | 158 | 167 |
| R-NTP | 152 | 176 |
| R-D&N | 166 | 150 |
| L-DTT | 156 | 157 |
| L-NTP | 161 | 168 |
| L-D&N | / | 153 |

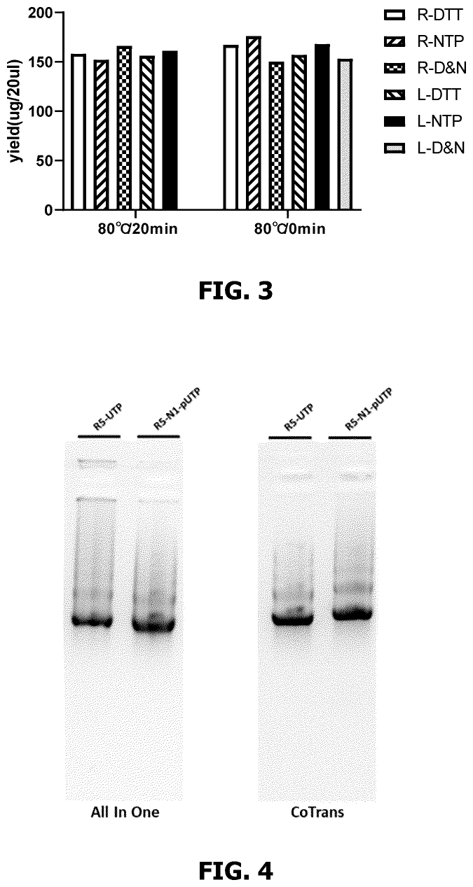

In the above, TABLE 4 is corresponding to data in FIG. 3.

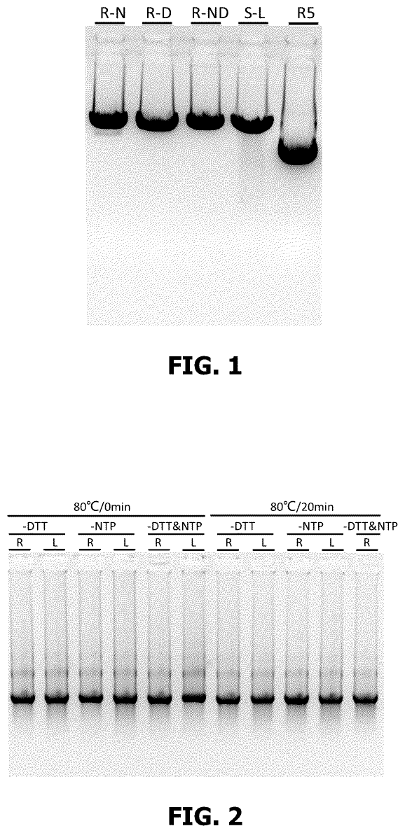

Experimental results are as shown in FIG. 1 to FIG. 3 and TABLE 4. Deletion of DTT and/or NTP in the linearization stage did not affect the linearization efficiency of the template and there was no remarkable difference in RNA yield; inactivating BspQI by heating after the linearization was ended had no obvious influence on the RNA yield/integrity.
2. The All In One system was compared with the conventional mRNA preparation process (taking novel coronavirus S protein as an example, NTP 7.5 m). Scheme 1 was preparation of mRNA through the All In One process, and scheme 2 was preparation of mRNA through a CoTrans (cotranscription) process.

### Experimental Steps

System configurations D and E are corresponding to TABLE 5 and TABLE 6 respectively.

TABLE 5
Scheme 2

| | | |
|---|---|---|
| Tris-Ac(pH 8.0; 1M) | 30 | mM |
| rATP(200 mM) | 7.5 | mM |
| rCTP(200 mM) | 7.5 | mM |
| rUTP(200 mM) | 7.5 | mM |
| rGTP(200 mM) | 1.5 | mM |
| CleanCap AG(100 mM) | 6 | mM |
| Template | 0.075 | μg/μL |
| MgAc₂(1M) | 40 | mM |
| Spermidine (100 mM) | 2.0 | mM |
| DTT(500 mM) | 20 | mM |
| IPP(100 U/mL) | 0.004 | U/μL |

| | |
|---|---|
| RNase Inhibitor(40000 U/mL) | 1 U/μL (0 U/μL-5 U/μL/0 U/μL- |
| | 5 U/μL) |
| T7 polymerase(50000 U/mL) | 5 U/μL (2.5 U/μL-10 U/μL/1.25 U/μL- |
| | 10 U/μL) |
| RNase free Water | to 20 μL |

TABLE 6
Scheme 1

| | | |
|---|---|---|
| Tris-Ac(pH 8.0; 1M) | 30 | mM |
| rATP(200 mM) | 7.5 | mM |
| rCTP(200 mM) | 7.5 | mM |
| rUTP/rN1-Me-pUTP(200 mM) | 7.5 | mM |
| rGTP(200 mM) | 7.5 | mM |
| CleanCap AG(100 mM) | 7.5 | mM |
| Template | 0.075 | μg/μL |
| MgAc₂(1M) | 40 | mM |
| CaCl₂(10 mM) | 0.5 | mM |
| Spermidine (100 mM) | 20 | mM |
| BspQI(10000 U/mL) | 0.2 | U/μL |

| | |
|---|---|
| RNase free Water | to 20 μL |

All of the above used the template R5 (SARS-CoV-2 Spike WT; 6028 bp) plasmid DNA, and RNase free water was added to render a final in vitro transcription system 20 μL (DTT/IPP/T7/RNase-In volume was removed in the linearization stage).
1) In Scheme 2, reaction was carried out at 37° C. for 3 h (operation steps of cotranscription system were carried out according to conventional operation steps); the system of scheme 1 was mixed uniformly, and was first placed at 50° C. for 1 h; at the end of reaction, 1 μL was taken to measure linearization efficiency, then DTT (500 mM) 0.8 μL, IPP (100 U/mL) 0.8 μL, RNase Inhibitor (40,000 U/mL) 0.5 μL, and T7 polymerase (50,000 U/mL) 2 μL were added in sequence;
2) resultant was placed at 37° C. for 3 h, added with 3 μL of DNase I at the end of reaction and mixed uniformly, and placed at 37° C. for 30 min;
3) at the end of reaction, a lithium chloride precipitation method was used; and
4) 1.5 μL of RNA sample was additionally taken, mixed with 9 μL of RNase free water and 2 μL of 6× loading buffer uniformly, and analyzed for purity/integrity/size by 1% non-denaturing agarose gel electrophoresis.

Statistic results of yields of mRNAs prepared by two processes are as follows.

TABLE 7
Statistics of Yields of mRNAs Prepared by Two Processes

| | UTP | N1-pUTP |
|---|---|---|
| Scheme 1 | 144.28 | 138.411 |
| Scheme 2 | 50.244 | 55.08 |

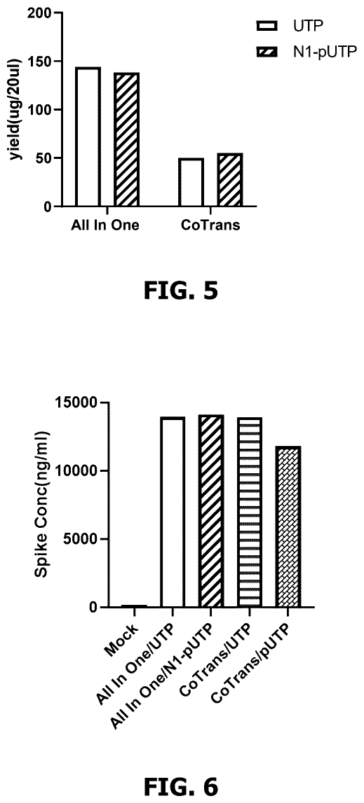

In the above, TABLE 7 is corresponding to data in FIG. 5.

TABLE 8
Expression of mRNAs Prepared by Two Processes after being
Electrotransferred to 293T Cells, analyzed by ELISA Assay

| | Scheme 1/ | Scheme 1/ | Scheme 2/ | Scheme 2/ |
|---|---|---|---|---|
| Mock | UTP | N1-pUTP | UTP | N1-pUTP |
| 215.95874 | 13939.28303 | 14120.6987 | 13922.75519 | 11816.10097 |

In the above, TABLE 8 is corresponding to data in FIG. 6.

Experimental result: mRNAs prepared by the processes of scheme 2 and scheme 1 had equal purity, and the mRNA purity was still similar when N1-Me-pUTP was used to replace rUTP.

As shown in FIG. 5 and TABLE 7, the process of scheme 1 had a slightly higher yield than that the cotranscription; and as shown in FIG. 6 and TABLE 8, the mRNA prepared by the process of scheme 1 had comparable expression level in 293T.

To sum up, the mRNA prepared by the process of scheme 1 (All In One) had the same purity as that of the mRNA prepared in scheme 2, and had a higher yield. The mRNA prepared by the process of scheme 1 (All In One) and the mRNA prepared by scheme 2, after electrotransformation into 293T cells, had comparable expression level of Spike protein.
3. orthogonal screening and comparison of Tris-Ac and Tris-HCl system (taking novel coronavirus S protein as an example, NTP 14 mM)

### Experimental Steps

System configuration is as shown in TABLE 9.

TABLE 9
All In One 4-Factor 3-Level Experiment, 9 Experiments

| Tris-Ac/Tris-HCl(pH 8.0; 1M) | 30 mM/40 Mm/50 mM | |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 40 Mm/50 Mm/60 mM | |
| CaCl₂(10 mM) | 0.5 | mM |
| Spermidine (100 mM) | 2.0 | mM |
| BspQI(10000 U/mL) | 0.2 | U/μL |

| | |
|---|---|
| RNase free Water | to 20 μL |

All of the above used a template: R39 (SARS-CoV-2 Spike WT; 6028 bp) plasmid DNA, and RNase free water was added to render a final in vitro transcription system 20 μL (DTT/IPP/T7/RNase-In volume was removed in the linearization stage). T7 promoter (transcription initiation site being AGG), R globin 5′UTR/3′UTR, SARS-Cov-2 Spike (NCBI Reference Sequence: NC_045512.2) and polyA were assembled together by a molecular biological method and finally ligated with a pUC57 cloning vector to form R39.
1) The system was mixed uniformly and placed at 50° C. for 1 h, and at the end of reaction, 1 μL was taken to measure linearization efficiency; and then DTT (500 mM) 0.8 μL/1 μL/1.2 μL, IPP (100 U/mL) 0.8 μL/1.25 μL/1.5 μL, RNase Inhibitor (40,000 U/mL) 0.5 μL, and T7 polymerase (50,000 U/mL) 2 μL were added in sequence;
2) resultant was placed at 37° C. for 3 h, added with 3 μL of DNase I at the end of reaction and mixed uniformly, and placed at 37° C. for 30 min;
purification A: lithium chloride precipitation method;
purification B: OligodT purification, performed by AKATA purifier with reference to instructions of POROS™ GoPure™ Oligo (dT)25 prepacked chromatographic column (Thermo Scientific); and
3) finally 1.5 μL of RNA sample was taken, mixed with 9 μL of RNase free water and 2 μL of 6× loading buffer uniformly, and analyzed for purity/integrity/size by 1% non-denaturing agarose gel electrophoresis; 5 μL was taken to analyze purity of mRNA by RPLC; 5 μg of respective purified mRNA was taken and subjected to electrotransfering with 2*10\hat{ }6 293T cells, and cultured in a 5% CO₂ incubator at 37° C. for 16 h; 100 μL of the cells were collected and lysed on ice with 2.5% SDS, and centrifuged at 13,000 g for 5 min, and supernatant was detected for S protein by ELISA.

TABLE 10
Expression of mRNA Prepared by Tris-Ac/Tris-HCl
Orthogonal System in 293T Cells by ELISA Assay

| | Tris-Ac | Tris-HCl |
|---|---|---|
| LiCl + EtOH | 1040.729 | 1075.239 |
| OligodT | 606.611 | 537.973 |

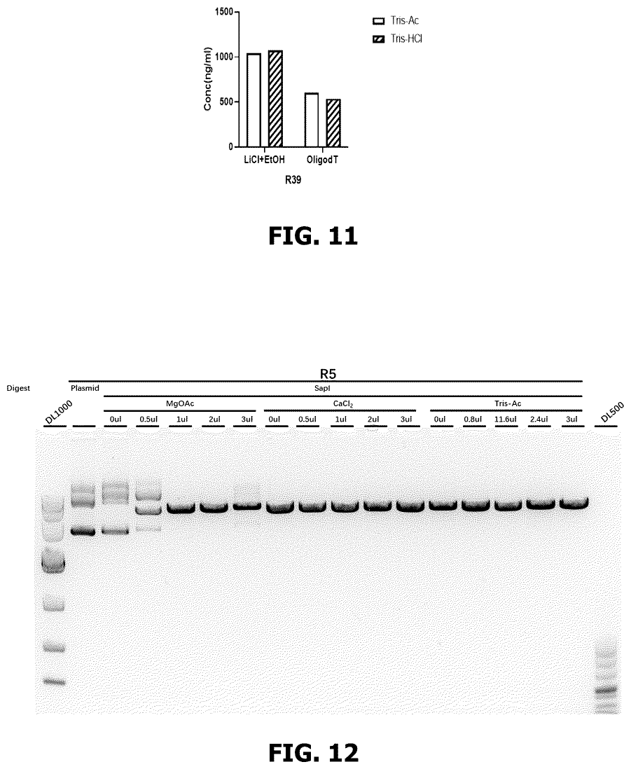

In the above, TABLE 10 is corresponding to data in FIG. 11.

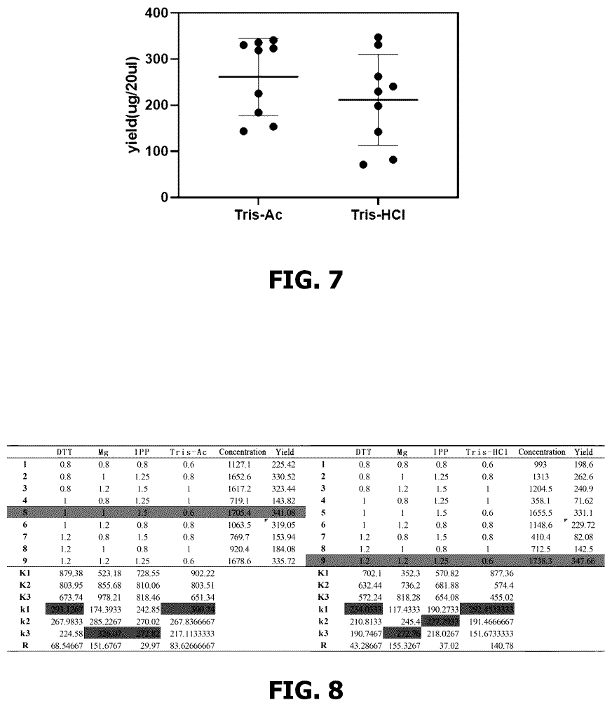

Experimental results are as shown in FIG. 7 and FIG. 8. Tris-Ac as a buffer enables, when other components are changed, a lower deviation of overall yield and a higher yield median line than that of Tris-HCl. As shown in FIG. 11 and TABLE 10, the mRNA prepared with Tris-Ac as buffer, upon lithium chloride precipitation purification or oligodT column chromatography purification, has translation efficiency in cells equivalent to that of Tris-HCl. Tris-Ac system has a higher yield and is more stable than Tris-HCl. Upon orthogonal screening of DTT/Mg/IPP/Tris concentration and fixing NTP at 14 mM, the yield obtained from final screening can be up to 350 μg/20 μL.

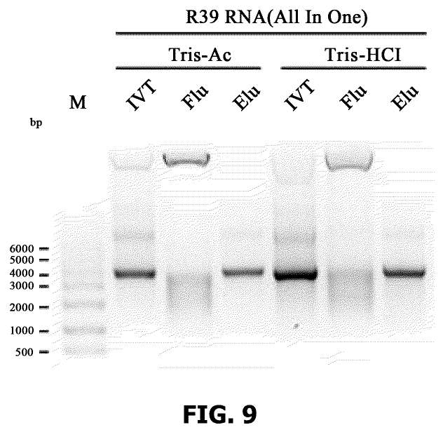

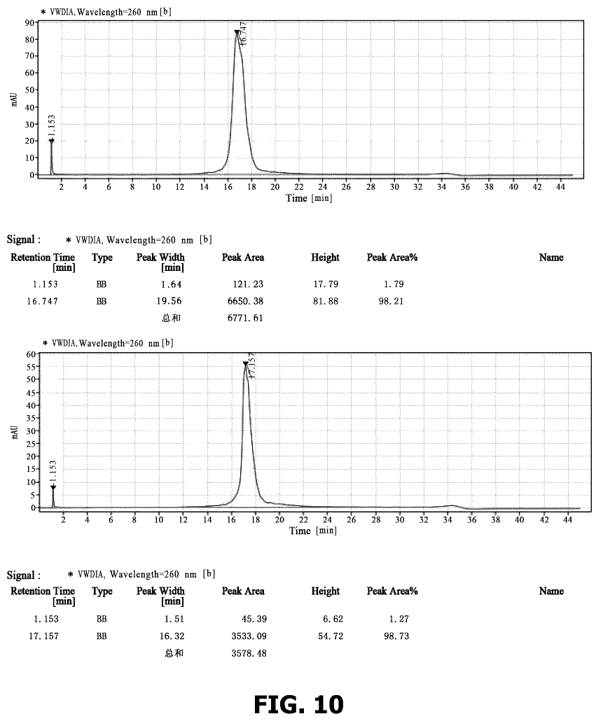

To sum up, Tris-Ac in different combinations renders a higher yield than Tris-HCl, and the mRNA prepared by the All In One system has the characteristics of high purity (as shown in FIG. 9) and good functional activity.

### Example 2 Characterization of Concentration Ranges of Components of all in One System and Component Replacement

### 1. Characterization of Concentration Ranges of Components of System

1.1.1 Characterization of Tris-Ac/MgAc₂/CaCl₂ Concentration

### Experimental Steps

System configuration is as shown in TABLE 11.

TABLE 11
All In One, 3-Factors 5-Levels

| Tris-Ac(pH 8.0; 1M) | 0 mM/40 mM/80 mM/120 mM/150 mM | |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 0 mM/25 mM/50 mM/100 mM/150 mM | |
| CaCl₂(10 mM) | 0 mM/0.25 mM/0.5 mM/1 mM/1.5 mM | |
| Spermidine (100 mM) | 2.0 | mM |
| BspQI(10000 U/mL) | 0.2 | U/μL |

| | |
|---|---|
| RNase free Water | to 20 μL |

In the above, there were 15 experiments in total, each having two tubes of replicates. All of the above used a template: R5 (SARS-CoV-2 Spike WT; 6028 bp) plasmid DNA, and RNase free water was added to render a final in vitro transcription system 20 μL (DTT/IPP/T7/RNase-In volume was removed in a linearization stage).
1) 2 tubes of repeating system were mixed uniformly, and then placed at 50° C. for 1 h; at the end of reaction, 1 μL was taken from one of the 2 tubes of repeating system to measure linearization efficiency; then DTT (500 mM) 0.8 μL, IPP (100 U/mL) 1.25 μL, RNase Inhibitor (40,000 U/mL) 0.5 μL, and T7 polymerase (50,000 U/mL) 2 μL were added in sequence and mixed uniformly, 10 μL was pipetted, added with 1.5 μL of DNase I, and placed at 37° C. for 30 min; at the end of reaction, 2 μL was taken for analysis of template digestion by agarose gel electrophoresis; remaining sample further reacted to 60 min, and a certain amount of sample was likewise taken for analysis of template digestion by agarose gel electrophoresis;
2) into one of the two tubes of repeating system, DTT (500 mM) 0.8 μL, IPP (100 U/mL) 1.25 μL, RNase Inhibitor (40,000 U/mL) 0.5 μL, and T7 polymerase (50,000 U/mL) 2 μL were added, mixed uniformly, and placed at 37° C. for 3 h, and at the end of reaction, 3 μL of DNase I was added, mixed uniformly, and placed at 37° C. for 30 min;
3) purification was performed by a lithium chloride precipitation method; and
4) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 12
Analysis of Influence of Different Tris-Ac/MgAc₂/
CaCl₂ Concentrations on Yield of in vitro Transcribed mRNA

| | | |
|---|---|---|
| Tris-Ac | MgOAc | CaCl₂ |

| | | | | | |
|---|---|---|---|---|---|
| 0 | 329.78 | 0 | 10.69 | 0 | 333.65 |
| 0.8 | 326.16 | 0.5 | 81.16 | 0.5 | 346.81 |
| 1.6 | 335.01 | 1 | 333.65 | 1 | 386.93 |
| 2.4 | 286.96 | 2 | 326.58 | 2 | 349.26 |
| 3 | 178.97 | 3 | 133.71 | 3 | 332.91 |

In the above, TABLE 12 is corresponding to data in FIG. 14.

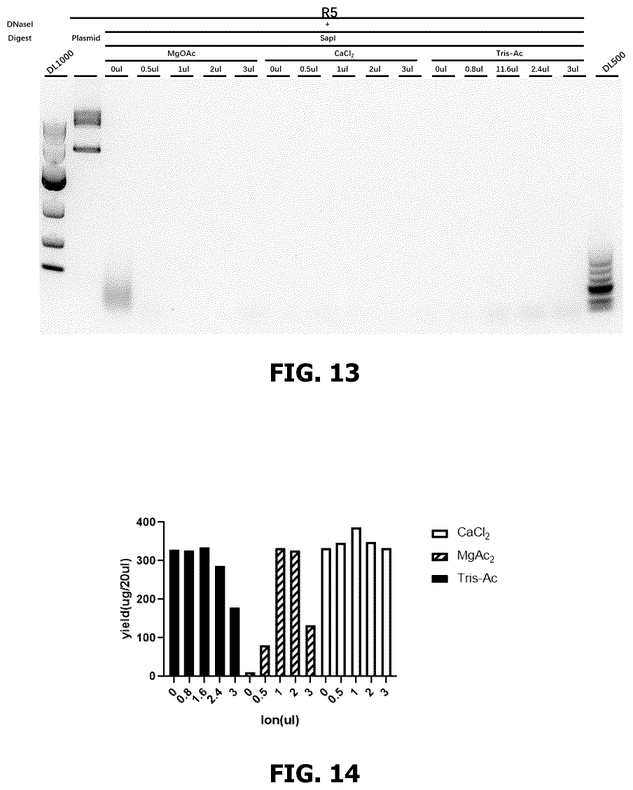

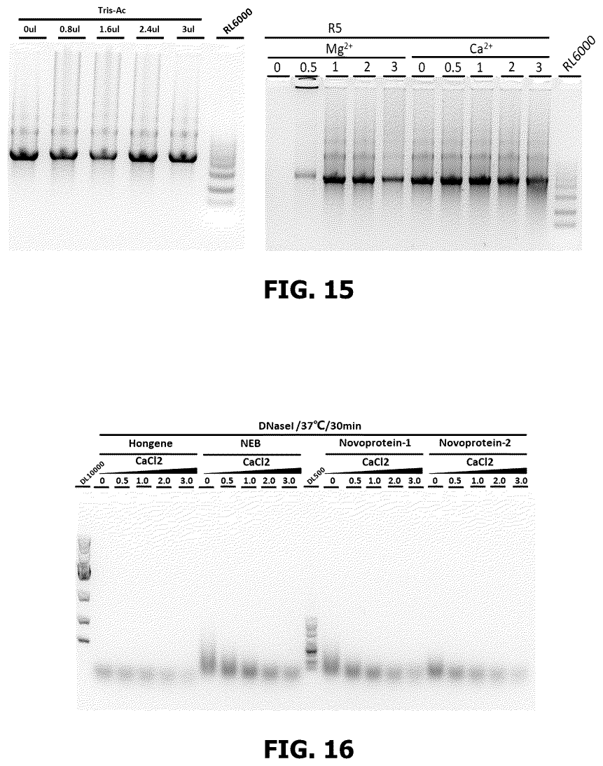

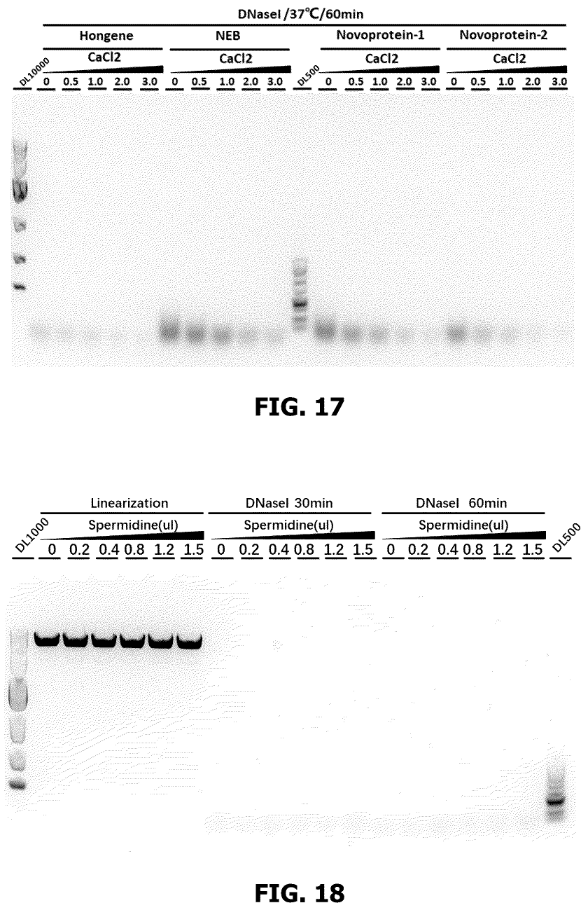

Experimental results are as shown in FIG. 12 to FIG. 15 and TABLE 12. CaCl₂ can realize about 100% linearization efficiency on plasmid. In order to degrade the template to 50 bp or below at 37° C. within 30 min, optimal CaCl₂ was (0.5 Mm-1.5 mM), the transcription yield likewise can reach 15 mg/mL; Tris-Ac can realize linearization on the plasmid and degrade the template to be 50 bp or below, and when a concentration range thereof was 0-120 mM, the in vitro transcription yield can be up to 15 mg/mL; MgAc₂ can realize linearization on the plasmid template (linearization efficiency was about 100% in a range of 50-150 mM), optimal MgAc₂ was (25-150 mM) in DNase I digestion, and in the in vitro transcription, when MgAc₂ was (25-150 mM), the mRNA yield was greater than 5 mg/mL (when MgAc₂ was (50-100 mM), the yield was up to 15 mg/mL).

### 1.1.2 Characterization of Activity of Different DNase I in Different CaCl₂

System configuration in experimental steps is as shown in TABLE 13.

TABLE 13
All In One

| Tris-Ac(pH 8.0; 1M) | 40 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0 mM/0.25 mM/0.5 mM/1 mM/1.5 mM | |
| Spermidine (100 mM) | 2.0 | mM |
| BspQI(10000 U/mL) | 0.2 | U/μL |

| | |
|---|---|
| RNase free Water | to 20 μL |

In the above, there were 20 experiments in total. All of the above used a template: R5 (SARS-CoV-2 Spike WT; 6028 bp) plasmid DNA, and RNase free water was added to render a final in vitro transcription system 20 μL (DTT/IPP/T7/RNase-In volume was removed in the linearization stage).
1) After uniform mixing, mixture was placed at 50° C. for 1 h, and added with DTT (500 mM) 0.8 μL, IPP (100 U/mL) 1.25 μL, RNase Inhibitor (40,000 U/mL) 0.5 μL, and T7 polymerase (50,000 U/mL) 2 μL in sequence and mixed uniformly, 10 μL was pipetted, added with 1.5 μL of DNase I (Hongene/Novoprotein/NEB) from different manufacturers, and placed at 37° C. for 30 min; at the end of reaction, 2 μL was taken for analysis of template digestion by agarose gel electrophoresis; remaining sample further reacted to 60 min, and a certain amount of sample was likewise taken for analysis of template digestion by agarose gel electrophoresis; and
2) 1 μL of RNA sample was taken, mixed with 5 μL of RNase free water and 1 μL of 6× loading buffer uniformly, and analyzed for purity/integrity/size by 1% non-denaturing agarose gel electrophoresis.

Experimental results indicate that by adding calcium chloride into the transcription system to test DNase I from different manufactures, it is found from the results that adding 1.0 μL of CaCl₂ facilitated DNase I in degrading starter transcription template.

### 1.2 Characterization of Spermidine Concentration

### Experimental Steps

System configuration is as shown in TABLE 14.

TABLE 14
All In One

| Tris-Ac(pH 8.0; 1M) | 40 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidine (100 mM) | 0 mM/1 mM/2 mM/4 mM/6 mM/7.5 mM | |
| BspQI(10000 U/mL) | 0.2 | U/μL |

| | |
|---|---|
| RNase free Water | to 20 μL |

In the above, there were 7 experiments in total, each having two replicates. All of the above used a template: R5 (SARS-CoV-2 Spike WT; 6028 bp) plasmid DNA, and RNase free water was added to render a final in vitro transcription system 20 μL (DTT/IPP/T7/RNase-In volume was removed in the linearization stage).
1) It was the same as step 1) and step 2) of 1.1.1 in Example 2, and sample was obtained and then purified by the lithium chloride precipitation method; and
2) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 15
Analysis of Influence of Different Spermidine Concentrations
on Yield of in vitro Transcribed mRNA

| 0 | 0.2 | 0.4 | 0.8 | 1.2 | 1.5 |
|---|---|---|---|---|---|
| 329.89 | 319.88 | 337.57 | 319.32 | 332.08 | 326.64 |

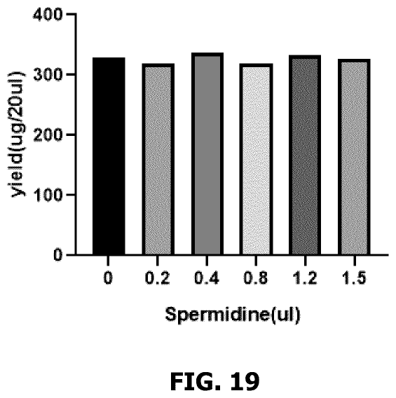

In the above, TABLE 15 is corresponding to data in FIG. 19; amounts added in TABLE 15 are corresponding to final concentrations in TABLE 14 (corresponding spermidine concentrations were set to be 0 mM/i mM/2 mM/4 mM/6 mM/7.5 mM; therefore, spermidine at 6 corresponding concentrations was added to 6 corresponding volumes in TABLE 15 (to satisfy a total volume of 20 μL).

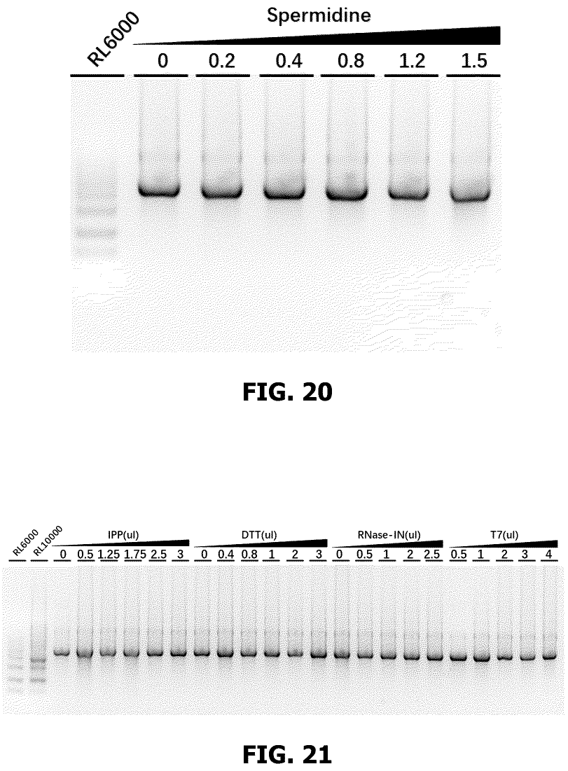

Experimental results are as shown in FIG. 18 to FIG. 20 and TABLE 15, and spermidine (0-7.5 mM) had no remarkable influence on template linearization/DNase I/in vitro transcription.

### 1.3 DTT/T7/IPP/RNase-Inhibitor Concentration Characterization

### Experimental Steps

System configuration is as shown in TABLE 16.

TABLE 16
All In One

| | | |
|---|---|---|
| Tris-Ac(pH 8.0; 1M) | 40 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidine (100 mM) | 2.0 | mM |
| BspQI(10000 U/mL) | 0.2 | U/μL |

| | |
|---|---|
| RNase free Water | to 20 μL |

In the above, there were 22 experiments in total, each having two replicates. All of the above used a template: R5 (SARS-CoV-2 Spike WT; 6028 bp) plasmid DNA, and RNase free water was added to render a final in vitro transcription system 20 μL (DTT/IPP/T7/RNase-In volume was removed in the linearization stage).
1) One tube was the same as in step 1) of 1.1.1 in Example 2;
2) 2 tubes of repeating system were each added with DTT (500 mM) 0 μL/0.4 μL/0.8 μL/1 μL/2 μL/3 μL, IPP (100 U/mL) 0 μL/0.5 μL/1.25 μL/1.75 μL/2.5 μL/3 μL, RNase Inhibitor (40,000 U/mL) 0 μL/0.5 μL/1 μL/2 μL/2.5 μL, and T7 polymerase (50,000 U/mL) 0 μL/0.5 μL/1 μL/2 μL/3 μL/4 μL and mixed uniformly at 37° C. for 3 h; at the end of reaction, 3 μL of DNase I was added and mixed uniformly, and placed at 37° C. for 30 min;
3) purification was performed by the lithium chloride precipitation method; and
4) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 17
Analysis of Influence of Different RNase-Inhibitor Enzyme
Volumes on Yield of in vitro Transcribed mRNA

| 0 μL(0 U) | 0.5 μL (20 U) | 1 μL (40 U) | 2 μL (80 U) | 2.5 μL (100 U) |
|---|---|---|---|---|
| 332.88 | 321.35 | 315.85 | 341.03 | 353.13 |

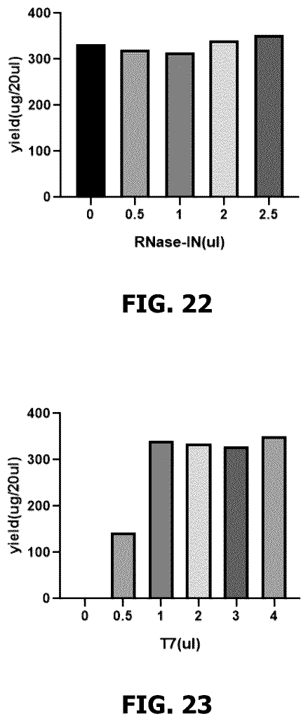

In the above, TABLE 17 is corresponding to data in FIG. 22.

TABLE 18
Analysis of Purity of in vitro Transcribed
mRNA with different T7 Enzyme Volumes

| 0 μL | 0.5 μL | 1 μL | 2 μL | 3 μL | 4 μL |
|---|---|---|---|---|---|
| (0 U) | (25 U) | (50 U) | (100 U) | (150 U) | (200 U) |
| 0.71 | 143.75 | 340.53 | 335.87 | 329.79 | 351.5 |

In the above, TABLE 18 is corresponding to data in FIG. 23.

TABLE 19
Analysis of Different IPP Volumes on
Purity of in vitro Transcribed mRNA

| 0 μL | 0.5 μL | 1.25 μL | 1.75 μL | 2.5 μL | 3 μL |
|---|---|---|---|---|---|
| (0 U) | (0.05 U) | (0.125 U) | (0.175 U) | (0.25 U) | (0.3 U) |
| 191.94 | 349.19 | 346.31 | 333.72 | 329.72 | 312.37 |

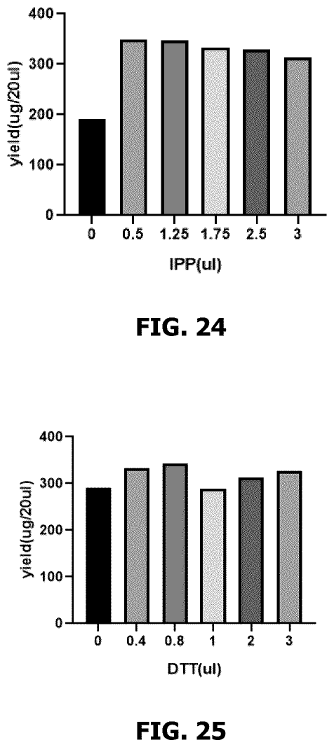

In the above, TABLE 19 is corresponding to data in FIG. 24.

TABLE 20
Analysis of Different DTT Volumes on
Purity of in vitro Transcribed mRNA

| 0 μL | 0.4 μL | 0.8 μL | 1 μL | 2 μL | 3 μL |
|---|---|---|---|---|---|
| (0 mM) | (10 mM) | (20 mM) | (25 mM) | (50 mM) | (75 mM) |
| 290.72 | 332.96 | 343.75 | 288.93 | 312.27 | 327.99 |

In the above, TABLE 20 is corresponding to data in FIG. 25.

Experimental results are as shown in FIG. 21 to FIG. 25 and TABLE 17 to TABLE 20. RNase-Inhibitor (0-100 U) had no remarkable influence on purity and yield of mRNA in the in vitro transcription stage, and the yield was up to 16 mg/mL; T7 (25-200 U) had no influence on the purity of mRNA in the in vitro transcription stage, and the yield was higher than 7.5 mg/mL, and up to 16 mg/mL at 50-200 U; IPP (0-0.3 U) had no influence on the purity of mRNA in the in vitro transcription stage, and the yield was 10 mg/mL or higher, up to 16 mg/mL at 0.05-0.3 U; DTT (0-75 mM) had no influence on the purity of mRNA in the in vitro transcription stage, and the yield was 15 mg/mL or higher.

### 1.4 Characterization of BspQI/INTP/Template Quantity and Concentration

### Experimental Steps

System configuration is as shown in TABLE 21.

TABLE 21
All In One

| Tris-Ac(pH 8.0; 1M) | 40 | mM |
|---|---|---|
| rATP(200 mM) | 2 mM/5 mM/7 mM/14 mM/16 mM/18 mM | |
| rCTP(200 mM) | 2 mM/5 mM/7 mM/14 mM/16 mM/18 mM | |
| rUTP(200 mM) | 2 mM/5 mM/7 mM/14 mM/16 mM/18 mM | |
| rGTP(200 mM) | 2 mM/5 mM/7 mM/14 mM/16 mM/18 mM | |
| CleanCap AG(100 mM) | 2 mM/5 mM/7 mM/14 mM/16 mM/18 mM | |
| Template | 0 μg/μL/0.025 μg/μL/0.05 μg/μL/0.075 μg/μL/ | |
| | 0.1 μg/μL/0.15 μg/μL/0.2 μg/μL | |
| MgAc₂(1M) | 50 | mM |
| CaCl₂(10 mM) | 0.5 | mM |
| Spermidine (100 mM) | 2.0 | mM |

| | |
|---|---|
| BspQI(10000 U/mL) | 0 U/μL/0.1 U/μL/0.2 U/μL/0.4 U/μL/0.6 U/μL/ |
| | 0.75 U/μL |
| RNase free Water | to 20 μL |

In the above, there were 19 experiments in total, each having two replicates. All of the above used a template: R5 (SARS-CoV-2 Spike WT; 6028 bp) plasmid DNA, and RNase free water was added to render a final in vitro transcription system 20 μL (DTT/IPP/T7/RNase-In volume was removed in the linearization stage).
1) It was the same as step 1) and step 2) of 1.1.1 in Example 2, and sample was obtained and then purified by the lithium chloride precipitation method; and
2) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 22
Analysis of Influence of Different BspQI
Concentrations on Yield of mRNA

| 0 U/μL | 0.1 U/μL | /0.2 U/μL | 0.4 U/μL | 0.6 U/μL | 0.75 U/μL |
|---|---|---|---|---|---|
| 215.34 | 346.54 | 332.57 | 318.8 | 319.4 | 332.34 |

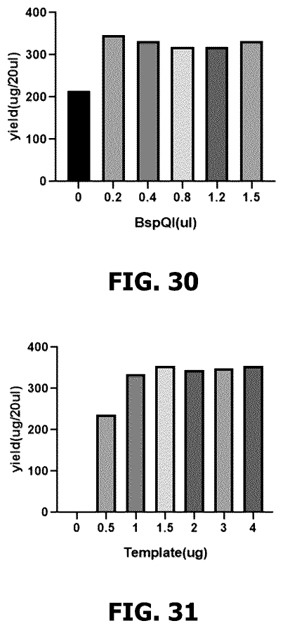

In the above, TABLE 22 is corresponding to data in FIG. 30; amounts added in TABLE 22 are corresponding to final concentrations in TABLE 21 (corresponding concentrations of BspQI were set to be 0 U/μL/0.1 U/μL/0.2 U/μL/0.4 U/μL/0.6 U/μL/0.75 U/μL; therefore, BspQI at 6 corresponding concentrations was added to 6 corresponding volumes in TABLE 22 (to satisfy a total volume of 20 μL).

TABLE 23
Analysis of Influence of Different Template R53 Concentrations on Yield of mRNA

| 0 μg/μL | 0.025 μg/μL | 0.05 μg/μL | 0.075 μg/μL | 0.1 μg/μL | 0.15 μg/μL | 0.2 μg/μL |
|---|---|---|---|---|---|---|
| 0.5 | 237.79 | 335.46 | 354.49 | 344.42 | 349.4 | 354.86 |

In the above, TABLE 23 is corresponding to data in FIG. 31.

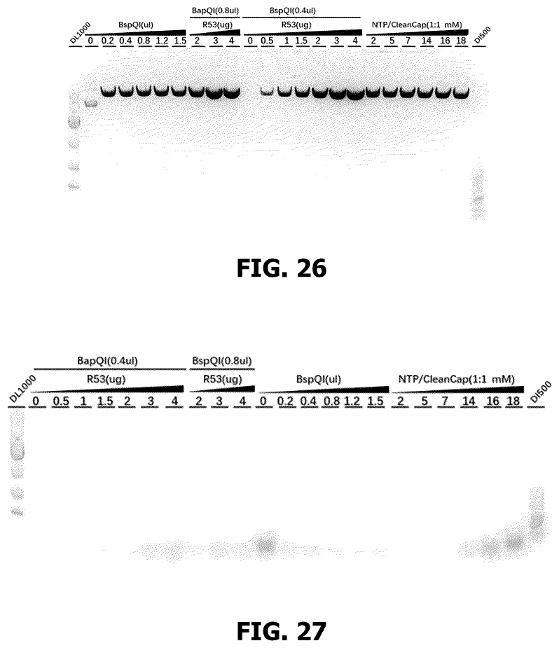

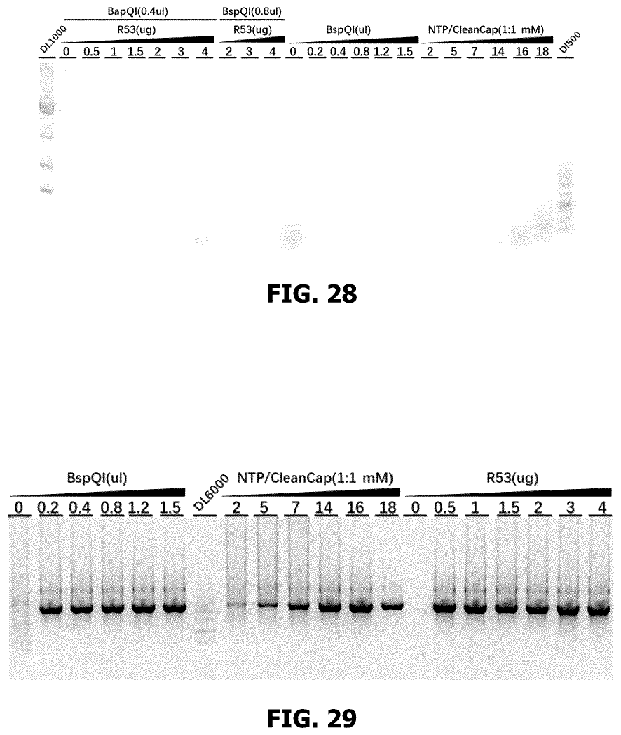

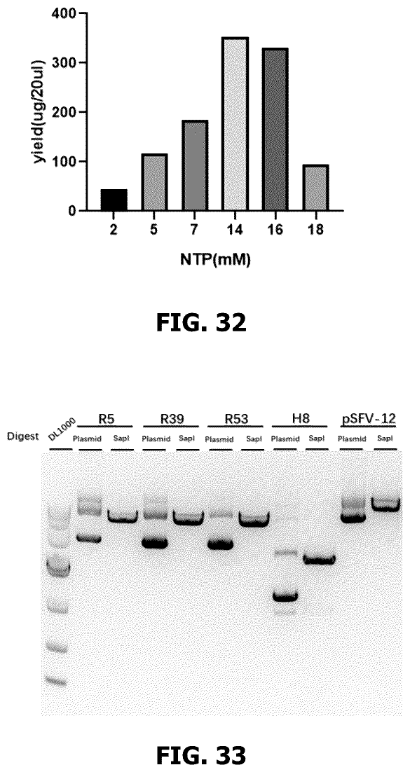

Experimental results are as shown in FIG. 26 to FIG. 32, TABLE 22 and TABLE 23. BspQI (2-15 U) had nearly 100% cleavage activity on 1.5 μg of template, and had no remarkable influence on the purity and yield of in vitro transcribed mRNA, and the yield was up to 16 mg/mL; the template quantities (0.025 μg/μL-0.2 μg/μL) can all be 100% cleaved by 4 U BspQI, the in vitro transcribed mRNA thereby had high purity, the yield was at least 11 mg/mL, and when the template quantity was (0.05 μg/μL-0.2 μg/μL), the yield can be up to 16 mg/mL; NTP/CleanCap (2-18 mM) had no influence on linearization, linearization efficiency was up to 100%, mRNA prepared by the in vitro transcription had high purity, and the lowest yield was about 2.5 mg/mL, and when the NTP/CleanCap was (14-16 Mm), the yield can be up to 16 mg/mL.

### 2. System Component Replacement Characterization

### 2.1 Template Replacement Characterization

### Experimental Steps

System configuration is as shown in TABLE 24.

TABLE 24
All In One

| | | |
|---|---|---|
| Tris-Ac(pH 8.0; 1M) | 40 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidine (100 mM) | 2.0 | mM |
| BspQI(10000 U/mL) | 0.2 | U/μL |

| | |
|---|---|
| RNase free Water | to 20 μL |

In the above, there were 5 experiments in total. All of the above used templates: R5/R39: SARS-CoV-2 Spike WT (6028 bp); R53: SARS-CoV-2 Spike Omicron (5951 bp); H18: HPV (2481 bp); and pSFV-12: SFV saRNA (11413 bp), and RNase free water was added to render a final in vitro transcription system 20 μL (DTT/IPP/T7/RNase-In volume was removed in the linearization stage).

T7 promoter (transcription initiation site being AGG), R globin 5′UTR/3′UTR, SARS-Cov-2 Omicron Spike and polyA were assembled together by a molecular biological method and finally ligated with a pUC57 cloning vector to form R53. Original structural protein of Semliki Forest virus isolate Tanzania53 (GenBank: MK280688.1) was replaced with eGFP by the molecular biological method, polyA was added to 3′UTR end of virus, T7 promoter was added to 5′UTR 5′-end and finally ligated with the pUC57 cloning vector to form pSFV12. T7 promoter (transcription initiation site being AG), R globin 5′UTR/3′UTR, Human papillomavirus type 16 E7 protein (Gene ID: 1489079) and polyA were assembled together by the molecular biological method and finally ligated with the pUC57 cloning vector to form H18.
1) After uniform mixing, mixture was placed at 50° C. for 1 h, and at the end of reaction, 1 μL was taken to measure linearization ZS efficiency; then DTT (500 mM) 0.8 μL, IPP (100 U/mL) 1.25 μL, RNase Inhibitor (40,000 U/mL) 0.5 μL, and T7 polymerase (50,000 U/mL) 2 μL were added in sequence and mixed uniformly, 10 μL was pipetted, added with 1.5 μL of DNase I, and placed at 37° C. for 30 min, and a certain amount of sample was taken for analysis of template digestion by agarose gel electrophoresis.

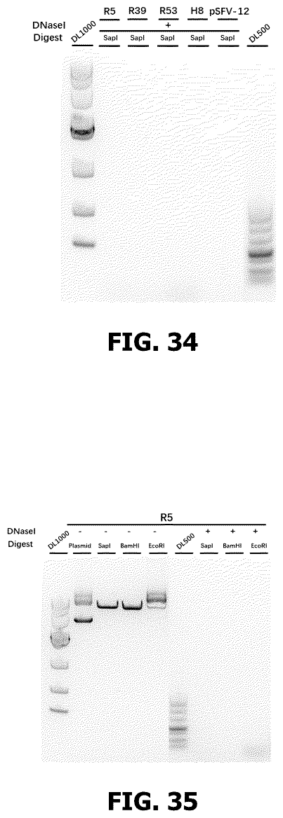

Experimental results are as shown in FIG. 33 and FIG. 34. Plasmid templates with different lengths or plasmid templates with the same length and different coding sequences can all be linearized in All In One and subsequently digested, further proving that the present experimental scheme system has the characteristic of wide adaptability.

### 2.2 Different Enzyme Replacement Characterization

### Experimental Steps

System configuration is as shown in TABLE 25.

TABLE 25
All In One

| | | |
|---|---|---|
| Tris-Ac(pH 8.0; 1M) | 40 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidine (100 mM) | 2.0 | mM |
| EcoRI/BamHI/BspQI(10000 U/mL) | 0.2 | U/μL |

| | |
|---|---|
| RNase free Water | to 20 μL |

In the above, there were 3 experiments in total. All of the above used a template: R5: SARS-CoV-2 Spike WT (6028 bp), and RNase free water was added to render a final in vitro transcription system 20 μL (DTT/IPP/T7/RNase-In volume was removed in the linearization stage), and digestion was carried out according to step 1) of 2.1 in Example 2.

Experimental results are as shown in FIG. 35. Different restriction endonucleases can linearize the R53 plasmid in the All In One, further proving that the present scheme system can be matched with restriction endonucleases other than BspQI.

### 2.3 Characterization of Different Ions Replacing MgAc₂

### Experimental Steps

System configuration is as shown in TABLE 26.

TABLE 26
All In One

| | | |
|---|---|---|
| Tris-Ac(pH 8.0; 1M) | 40 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidinc (100 mM) | 2.0 | mM |
| BspQI(10000 U/mL) | 0.2 | U/μL |

| | |
|---|---|
| RNase free Water | to 20 μL |

In the above, there were 8 experiments in total, each having two replicates. All of the above used a template: R5 (SARS-CoV-2 Spike WT; 6028 bp) plasmid DNA, the above MgAc₂ (1 M) was replaced with 1 M MnCl₂, MnAc₂, ZnSO₄, CaAc₂, CaCl₂, MgCl₂ and FeCl₃ in sequence, and RNase free water was added to render a final in vitro transcription system 20 μL (DTT/IPP/T7/RNase-In volume was removed in the linearization stage).
1) It was the same as step 1) and step 2) of 1.1.1 in Example 2, and sample was obtained and then purified by the lithium chloride precipitation method; and
2) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 27
Influence of Replacing MgAc₂ with Different Metal
Ions on Yield of in vitro transcribed mRNA

| MnCl₂ | MnAc₂ | ZnSO₄ | CaAc₂ | CaCl₂ | MgCl₂ | FeCl₃ | MgAc₂ |
|---|---|---|---|---|---|---|---|
| 36.25 | 57.41 | 76.2 | 9.17 | 7.37 | 235.2 | 154.45 | 347.73 |

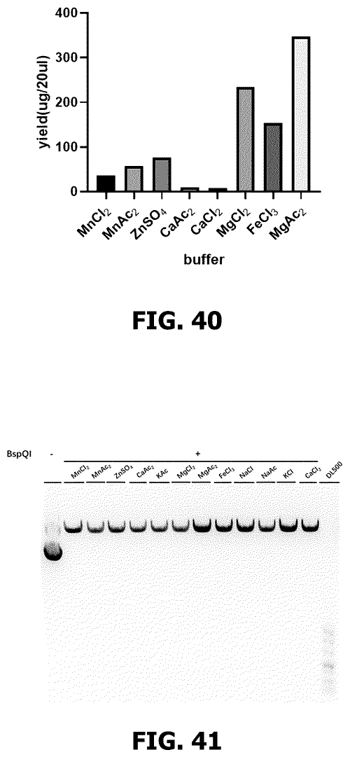

In the above, TABLE 27 is corresponding to data in FIG. 40.

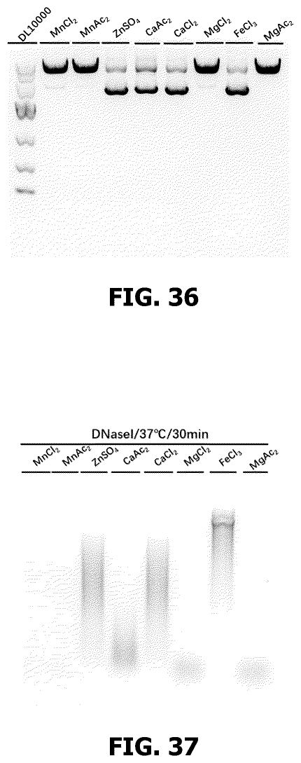

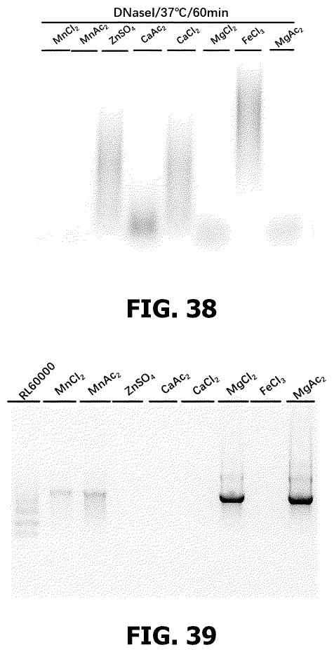

Experimental results are as shown in FIG. 36 to FIG. 40 and TABLE 27. In the linearization stage: MnCl₂, MnAc₂, ZnSO₄, CaAc₂, CaCl₂, MgCl₂ and FeCl₃ can all cleave the template, but cleavage activities of MnCl₂, MnAc₂ and MgCl₂ were close to MgAc₂, about 100%; in the stage of DNase I digesting template: DNase I can digest the template in the presence of MnCl₂, MnAc₂, ZnSO₄, CaAc₂, CaCl₂, MgCl₂ and FeCl₃, but the presence of MnCl₂, MnAc₂, CaAc₂ and MgCl₂ can greatly promote degradation of the template, especially in the presence of MnCl₂, MnAc₂ and MgCl₂, activity of DNase I was close to that of MgAc₂; in the in vitro transcription stage: by replacing MgAc₂ with MnCl₂, MnAc₂ and MgCl₂, mRNA can be prepared in the All In One, and the yield was about 2.5 mg/mL, where the yield can be increased to 11 mg/mL after the replacement with MgCl₂.

To sum up, in the whole system of the present scheme, in a case where only magnesium ions, especially magnesium acetate, were used, high yield mRNA can be prepared.

### 2.4 Characterization of Different Ion Replacing CaCl₂

### Experimental Steps

System configuration is as shown in TABLE 28.

TABLE 28
All In One

| | | |
|---|---|---|
| Tris-Ac(pH 8.0; 1M) | 40 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidine (100 mM) | 2.0 | mM |
| BspQI(10000 U/mL) | 0.2 | U/μL |

| | |
|---|---|
| RNase free Water | to 20 μL |

In the above, there were 12 experiments in total, each having two replicates. All of the above used a template: R5 (SARS-CoV-2 Spike WT; 6028 bp) plasmid DNA, the above CaCl₂) (10 mM) was replaced with 10 mM MnCl₂, MnAc₂, ZnSO₂, CaAc₂, CaCl₂, MgCl₂, NaAc, NaCl, KCl, KAc and FeCl₂ in sequence, and RNase free water was added to render a final in vitro transcription system 20 μL (DTT/IPP/T7/RNase-In volume was removed in the linearization stage).
1) It was the same as step 1) and step 2) of 1.1.1 in Example 2, and sample was obtained and then purified by the lithium chloride precipitation method; and
2) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 29
Influence of Replacing CaCl₂ with Different
Metal Ions on Yield of mRNA

| MnCl₂ | MnAc₂ | ZnSO₄ | CaAc₂ | KAc | MgCl₂ |
|---|---|---|---|---|---|
| 325.88 | 327.97 | 345.55 | 348.56 | 333.92 | 338.13 |
| MgAc₂ | FeCl₃ | NaCl | NaAc | KCl | CaCl₂ |
| 337.17 | 328.35 | 337.07 | 344.46 | 333.45 | 348.03 |

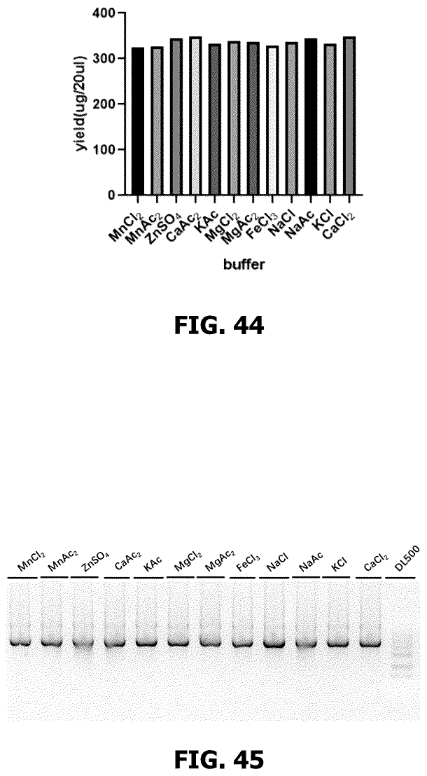

In the above, TABLE 29 is corresponding to data in FIG. 44.

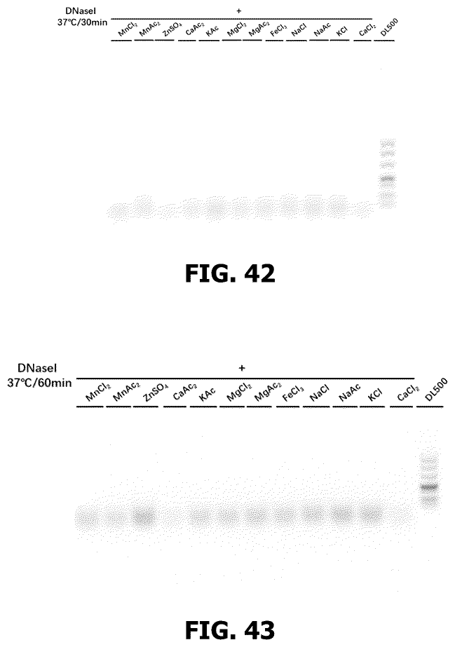

Experimental results are as shown in FIG. 41 to FIG. 44 and TABLE 29. In the linearization stage: replacing CaCl₂ with MnCl₂, MnAc₂, ZnSO₄, CaAc₂, CaCl₂, MgCl₂, NaAc, NaCl, KCl, KAc and FeCl₃ can cleave the template and cutting activity was about 100%; in the stage of DNase I digesting template: replacing CaCl₂ with MnCl₂, MnAc₂, ZnSO₄, CaAc₂, CaCl₂, MgCl₂, NaAc, NaCl, KCl, KAc and FeCl₃ can digest and degrade the template to 50 bp or below; in the in vitro transcription stage: replacing CaCl₂ with MnCl₂, MnAc₂, ZnSO₄, CaAc₂, CaCl₂, MgCl₂, NaAc, NaCl, KCl, KAc and FeCl₃ can all prepare high-purity mRNA and the yield was up to 15 mg/mL or above.

To sum up, for the whole preparation process of RNA, and in conjunction with FIG. 43 and FIG. 44, it can be further proved that addition of calcium ions enables DNase I to degrade residual template more thoroughly and render a higher yield of mRNA prepared.

### 2.5 Characterization of Different Buffer Replacement

### Experimental Steps

System configuration is as shown in TABLE 30.

TABLE 30
All In One

| | | |
|---|---|---|
| Tris-Ac(pH 8.0; 1M) | 40 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| Template | 0.075 | μg/μL |
| MgAc₂(1M) | 50 | mM |
| CaCl₂(10 mM) | 0.5 | mM |
| Spermidine (100 mM) | 2.0 | mM |
| BspQI(10000 U/mL) | 0.2 | U/μL |

| | |
|---|---|
| RNase free Water | to 20 μL |

In the above, there were 7 experiments in total, each having two replicates. All of the above used a template: R5 (SARS-CoV-2 Spike WT; 6028 bp) plasmid DNA, the above Tris-Ac (pH 8.0; 1 M) was replaced with pH 8.0; 1 M HEPES/KOH, Tris-MES, Tris-HCl, Tris-Ac, K₂HPO₄/KH₂PO₄, K₂HPO₄/NaH₂PO₄, and MOPS-Ac in sequence, and RNase free water was added to render a final in vitro transcription system 20 μL (DTT/IPP/T7/RNase-In volume was removed in the linearization stage).
1) It was the same as step 1) and step 2) of 1.1.1 in Example 2, and sample was obtained and then purified by the lithium chloride precipitation method; and
2) 1.5 μL of RNA sample was taken, mixed with 9 μL of RNase free water and 2 μL of 6× loading buffer uniformly, and analyzed for purity/integrity/size by 1% non-denaturing agarose gel electrophoresis.

TABLE 31
Influence of Replacing Tris-HCl with
Different Buffers on Yield of mRNA

| 1# | 2# | 3# | 4# | 5# | 6# | 7# |
|---|---|---|---|---|---|---|
| 317.28 | 345.31 | 349.36 | 343.96 | 354.67 | 333.03 | 330.19 |

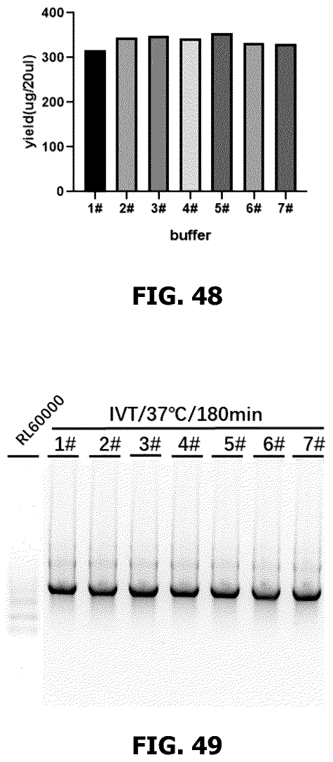

In the above, TABLE 31 is corresponding to data in FIG. 48.

In the above, all of 1 #HEPES/KOH, 2 #Tris-MES, 3 #Tris-HCl, 4 #Tris-Ac, 5 #K₂HPO₄/KH₂PO₄, 6 #K₂HPO₄/NaH₂PO₄, and 7 #MOPS-Ac were 1 M (Ph 8.0).

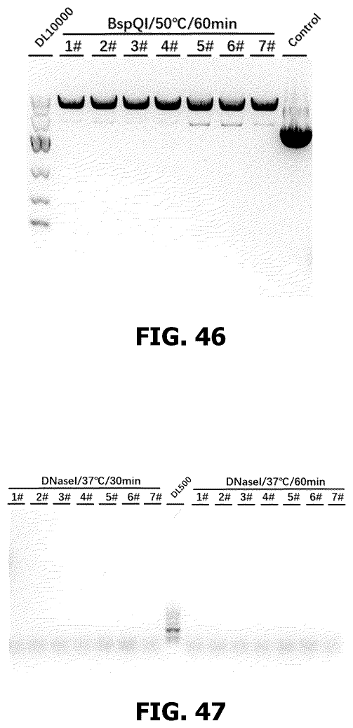

Experimental results are as shown in FIG. 46 to FIG. 49 and TABLE 31. In the linearization stage: HEPES/KOH, Tris-MES, Tris-HCl, K₂HPO₄/KH₂PO₄, K₂HPO₄/NaH₂PO₄, and MOPS-Ac, replacing Tris-Ac, can cleave the template; in the stage of DNase I digesting template: HEPES/KOH, Tris-MES, Tris-HCl, K₂HPO₄/KH₂PO₄, K₂HPO₄/NaH₂PO, and MOPS-Ac, replacing Tris-Ac, can cleave the template, and can digest and degrade the template to 50 bp or below; in the in vitro transcription stage: by replacing Tris-Ac with HEPES/KOH, Tris-MES, Tris-HCl, K₂HPO₄/KH₂PO₄, K₂HPO₄/NaH₂PO, and MOPS-Ac, high-purity mRNA can be prepared, and the yield was up to 15 mg/mL.

In conclusion, it can be seen in conjunction with FIG. 46 that Tris-Ac can be replaced by the above buffers to some extent, but considering that the linearization efficiency is a quality control term, Tris-Ac can make the linearization of the plasmid more thorough.

### Example 3 Phosphate Universal System

### 1. Test of Comparison Between Different Systems

Experimental steps: an in vitro transcription system except buffer was configured according to components in TABLE 32, and after uniform mixing, 19.4 μL was dispensed into 9 PCR tubes.

TABLE 32
In vitro Transcription System Except Buffer

| rATP(200 mM) | 14 | mM |
|---|---|---|
| rCTP(200 mM) | 14 | mM |
| rUTP(200 mM) | 14 | mM |
| rGTP(200 mM) | 14 | mM |
| CleanCap AG(100 mM) | 14 | mM |
| R53 (Linearized template) | 0.0675 | μg/μl |
| MgAc₂(1M) | 50 | mM |
| CaCl₂(10 mM) | 0.5 | mM |
| Spermidine (100 mM) | 2.0 | mM |
| RNase free Water | up to 20 μL | |
| IPP(100 U/mL) | 0.0075 | U/μL |
| DTT(500 mM) | 25 | mM |
| RNase Inhibitor(40000 U/mL) | 1 | U/μL |
| T7 polymerase(50000 U/mL) | 5 | U/μL |

All of the above used a template: R53 (Linearized template) (SARS-CoV-2 Spike Omocron; 5951 bp) plasmid DNA, 0.6 μL of 1 M Tris-Ac (pH 8.0), 1 M HEPES/KOH (pH 8.0), 1 M Tris-HCl (pH 8.0), 1 M K₂HPO₄/KH₂PO₄ (pH 8.0), 1 M K₂HPO₄/NaH₂PO₄ (pH 8.0), and 2 M HEPES/KOH (pH 7.5) were added in sequence, mixed uniformly and placed at 37° C. for 3 h. At the end of reaction, 3 μL of DNase I was added, mixed uniformly, placed at 37° C. for 30 min. Purification was performed by a lithium chloride precipitation method. 3 μg was taken and electrotransferred by 4D-Nucleofector. Cells were collected after 16 h, lysed with RIPA lysis solution, and centrifuged at 4° C. to collect supernatant for ELISA detection.

TABLE 33
Translation Efficiency of mRNAs Prepared by Different Systems

| | |
|---|---|
| 1M HEPES-KOH(pH 8.0) | 1176.29 |
| 1M K₂HPO₄/KH₂PO₄(pH 8.0) | 1330.89 |
| 1M K₂HPO₄/NaH₂PO₄(pH 8.0) | 1477.5 |
| 1M Tris-HCl(pH 8.0) | 1172.33 |
| 1M Tris-Ac(pH 8.0) | 1014.94 |
| 2M HEPES-KOH(pH 7.5) | 948.45 |

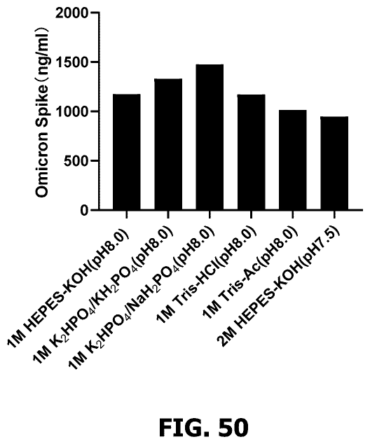

Experimental results are as shown in FIG. 50 and TABLE 33. It is found from the results that the mRNAs prepared by two phosphate systems have higher translation efficiency than other common systems.

### 2. Test of Optimal pH of Different Phosphate Systems

Sodium and potassium salts were combined to configure four different combined phosphate buffers: K₂HPO₄/KH₂PO₄, K₂HPO₄/NaH₂PO₄, Na₂HPO₄/KH₂PO₄ and Na₂HPO₄/NaH₂PO₄ were tested for respective optimal pH, where Na₂HPO₄/KH₂PO₄ was found to have obvious precipitate in formulation and thus was discarded.

Components were formulated as shown in TABLE 34.

TABLE 34
Different Combined Phosphate Buffers

| buffer | 45 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| R53-S | 0.0675 | μg/μL |
| MgAc₂(1M) | 50 | mM |
| CaCl₂(10 mM) | 0.5 | mM |
| Spermidine (100 mM) | 2.0 | mM |
| RNase free Water | up to 20 μL | |
| IPP(100 U/mL) | 0.0075 | U/μL |
| DTT(500 mM) | 25 | mM |
| RNase Inhibitor(40000 U/mL) | 1 | U/μL |
| T7 polymerase(50000 U/mL) | 5 | U/μL |

All of the above used a template: R53 (SARS-CoV-2 Spike Omocron; 5951 bp) plasmid DNA, 0.6 μL of 1 M K₂HPO₄/KH₂PO₄ (pH 7.2-8.0), 1 M K₂HPO₄/NaH₂PO₄ (pH 7.2-8.2) and 1M Na₂HPO₄/NaH₂PO₄ (pH 7.-7.8) were added in sequence, mixed uniformly and placed at 37° C. for 3 h. At the end of reaction, 3 μL of DNase I was added, mixed uniformly, placed at 37° C. for 30 min. Purification was performed by a lithium chloride precipitation method. 3 μg was taken and electrotransferred by 4D-Nucleofector. Cells were collected after 16 h, lysed with RIPA lysis solution, and centrifuged at 4° C. to collect supernatant for ELISA detection.

TABLE 35
Translation Efficiency of RNAs Prepared
by K₂HPO₄/KH₂PO₄ with Different pH

| | | |
|---|---|---|
| pH 7.0 | 676.69 | 1558.01 |
| pH 7.2 | 608.67 | 1234.66 |
| pH 7.4 | 1041.25 | 1647.47 |
| pH 7.5 | 1106.99 | 1826.75 |
| pH 7.6 | 1079.2 | 1895.87 |
| pH 7.8 | 1189.81 | 1844.03 |

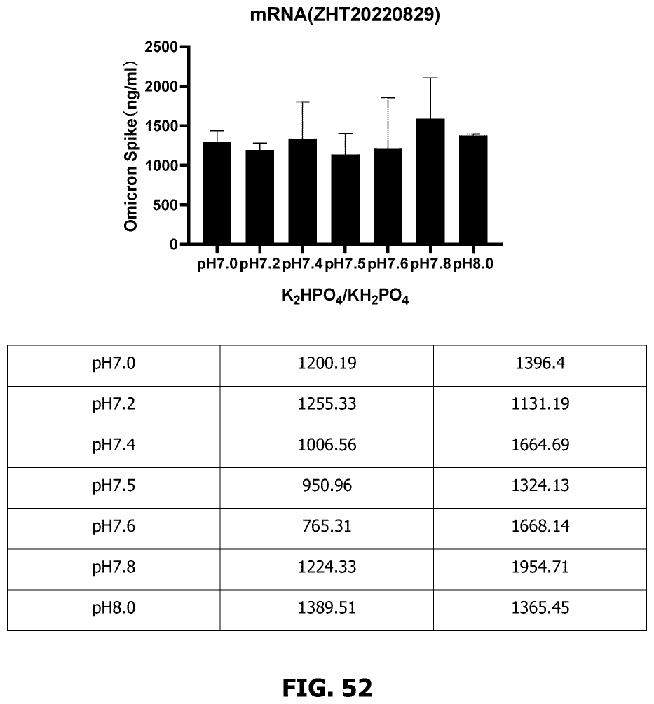

TABLE 35 is corresponding to data in FIG. 52.

TABLE 36
Translation Efficiency of RNAs Prepared
by Na₂HPO₄/NaH₂PO₄ with Different pH

| | | |
|---|---|---|
| pH 7.0 | 1200.19 | 1396.4 |
| pH 7.2 | 1255.33 | 1131.19 |
| pH 7.4 | 1006.56 | 1664.69 |
| pH 7.5 | 950.96 | 1324.13 |
| pH 7.6 | 765.31 | 1668.14 |
| pH 7.8 | 1224.33 | 1954.71 |
| pH 8.0 | 1389.51 | 1365.45 |

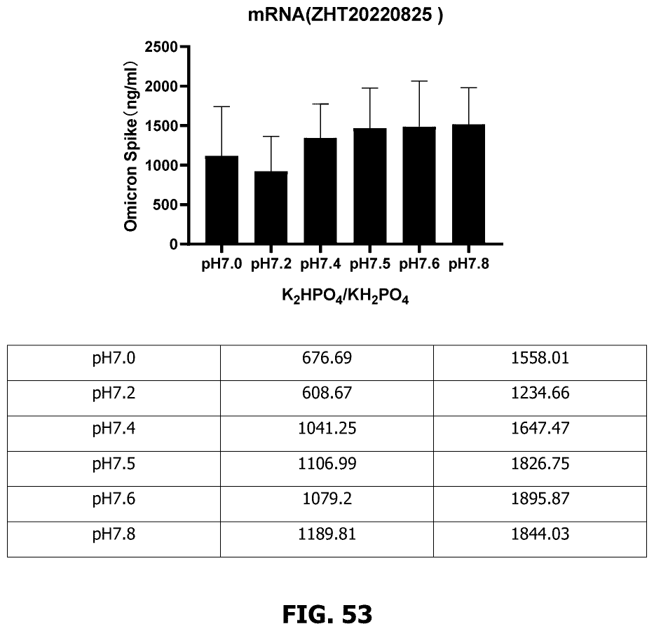

TABLE 36 is corresponding to data in FIG. 53.

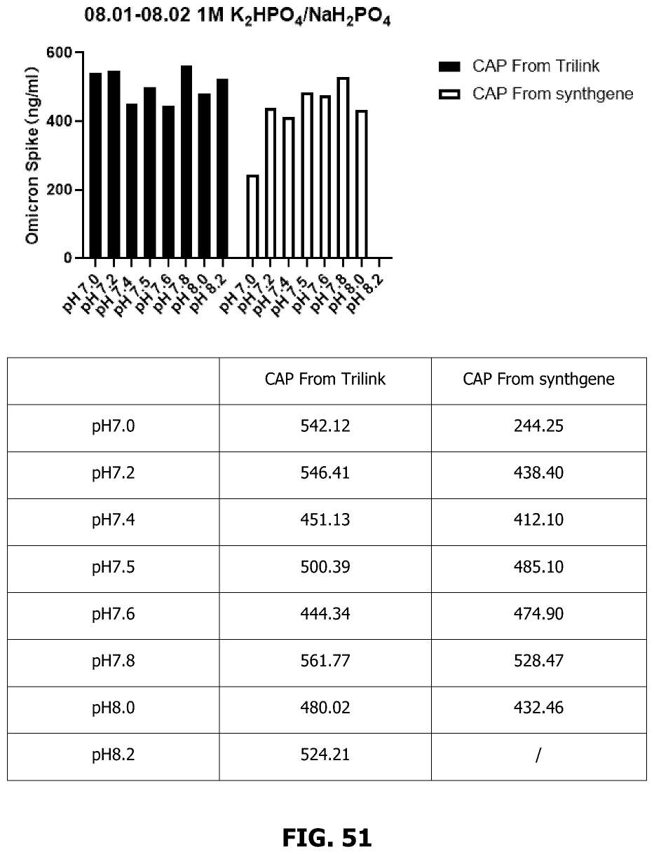

Experimental results are as shown in FIG. 51 to FIG. 53, TABLE 35 and TABLE 36. Optimal pH of K₂HPO₄/NaH₂PO₄ in the transcription system was 7.8; optimal pH of K₂HPO₄/KH₂PO₄ in the system was 7.8; and optimal pH of Na₂HPO₄/NaH₂PO₄ in the system was 7.8. But when K₂HPO₄/NaH₂PO₄ and Na₂HPO₄/NaH₂PO₄ were placed in a refrigerator at 4° C., salt precipitated occasionally, but it was soluble when heated.

3. Test of Optimal Concentration of K₂HPO₄/KH₂PO₄

Since a phenomenon of salt precipitation occurred occasionally when K₂HPO₄/NaH₂PO₄ and Na₂HPO₄/NaH₂PO₄ were placed in the refrigerator at 4° C., concentration of the K₂HPO₄/KH₂PO₄ system was temporarily measured.

Components were formulated as shown in TABLE 37.

TABLE 37
Test of Concentration of K₂HPO₄/KH₂PO₄ System

| K₂HPO₄/KH₂PO₄ | 15~75 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| R53-S | 0.0675 | μg/μL |
| MgAc₂(1M) | 50 | mM |
| CaCl₂(10 mM) | 0.5 | mM |
| Spermidine(100 mM) | 2.0 | mM |
| RNase free Water | up to 20 μL | |
| IPP(100 U/mL) | 0.0075 | U/μL |
| DTT(500 mM) | 25 | mM |
| RNase Inhibitor(40000 U/mL) | 1 | U/μL |
| T7 polymerase(50000 U/mL) | 5 | U/μL |

All of the above used the template: R53 (SARS-CoV-2 Spike Omocron; 5951 bp) plasmid DNA, 0.3 μL (15 mM), 0.6 μL (30 mM), 0.9 μL (45 mM), 1.2 μL (60 mM) and 1.5 μL (75 mM) of 1 M K₂HPO₄/KH₂PO₄ (pH 7.8) was added in sequence, and at the same time, groups added with 0.6 L (30 mM) of Tris-HCl, Tris-Ac and K₂HPO₄/NaH₂PO₄ were set, mixed uniformly and placed at 37° C. for 3 h. At the end of reaction, 3 μL of DNase I was added, mixed uniformly and placed at 37° C. for 30 min; at the same time, dsRNA residue was detected by DotBlot, purity analysis was performed by HPLC, and purification was performed by the lithium chloride precipitation method.

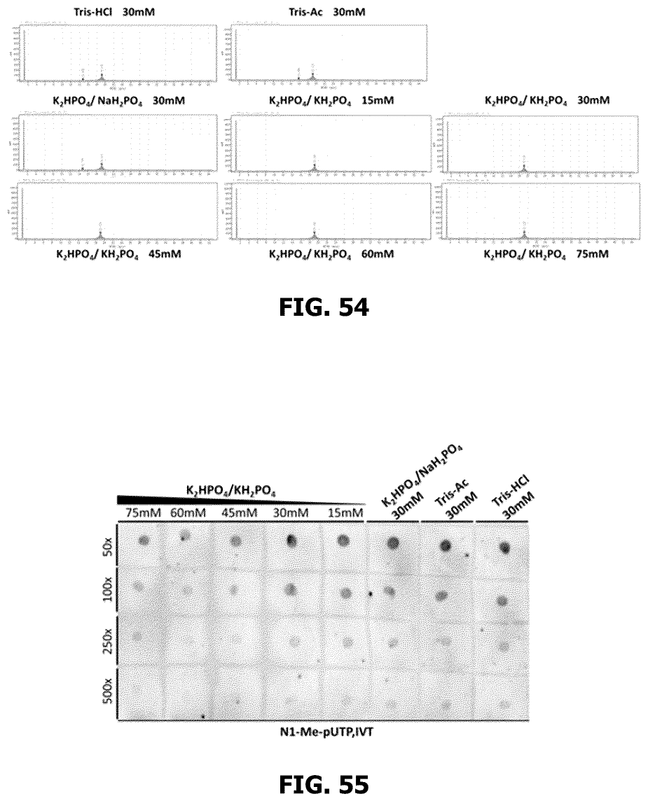

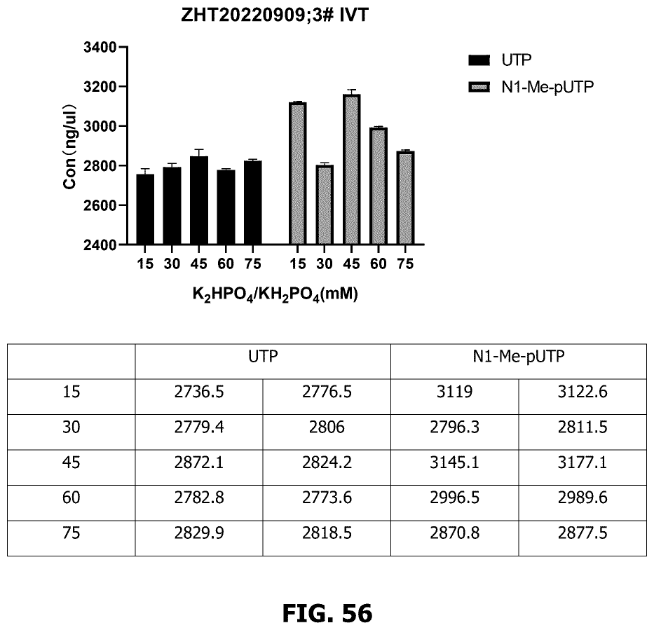

Experimental results are as shown in FIG. 54 to FIG. 56. The results indicate that the optimal concentration of K₂HPO₄/KH₂PO₄ (pH 7.8) in the transcription system was 45 mM, and the yield of mRNA at this concentration can reach μg level when taking UTP as a substrate; the yield can be up to μg level with modified nucleoside Ni-Me-pUTP; at the same time, by analyzing an unpurified reaction solution of mRNA by HPLC, it is found that K₂HPO₄/NaH₂PO₄, Tris-HCl and Tris-Ac as buffer system all had an impurity peak other than a main peak, while K₂HPO₄/KH₂PO₄ buffer system at different concentrations did not have this impurity peak. Finally, it is found by DotBlot analysis that 45-60 mM K₂HPO₄/KH₂PO₄ as buffer had lower dsRNA generation than the other groups, and thus K₂HPO₄/KH₂PO₄ was chosen as buffer for further development.

4. Adaptability of T7 Enzyme in K₂HP₄/KH₂PO₄ System

In view of the fact that phosphate is a novel transcription system, the present study hereby characterizes an optimal enzyme quantity, reaction duration and translation efficiency influence of T7 enzyme under this system.

Components were formulated as shown in TABLE 38.

TABLE 38
Adaptability of T7 Enzyme in K₂HPO₄/KH₂PO₄ System

| K₂HPO₄/KH₂PO₄ | 45 | mM |
|---|---|---|
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| R53-S | 0.0675 | μg/μL |
| MgAc₂(1M) | 50 | mM |
| CaCl₂(10 mM) | 0.5 | mM |
| Spermidine (100 mM) | 2.0 | mM |
| RNase free Water | up to 20 μL | |
| IPP(100 U/mL) | 0.0075 | U/μL |
| DTT(500 mM) | 25 | mM |
| RNasc Inhibitor(40000 U/mL) | 1 | U/μL |

| | |
|---|---|
| T7 polymerase(50000 U/mL) | 1.25 U/μL/2.5 U/μL/5 U/μL/7.5 U/μL |

All of the above used the template: R53 (SARS-CoV-2 Spike Omocron; 5951 bp) plasmid, which was mixed uniformly and placed at 37° C. for 0.5 h/1 h/2 h/3 h, respectively; at the end of reaction, 3 μL of DNase I was added, uniformly mixed, and placed at 37° C. for 30 min. Purification was performed by the lithium chloride precipitation method. 3 μg was taken and electrotransferred by 4D-Nucleofector. Cells were collected after 16 h, lysed with RIPA lysis solution, and centrifuged at 4° C. to collect supernatant for ELISA detection.

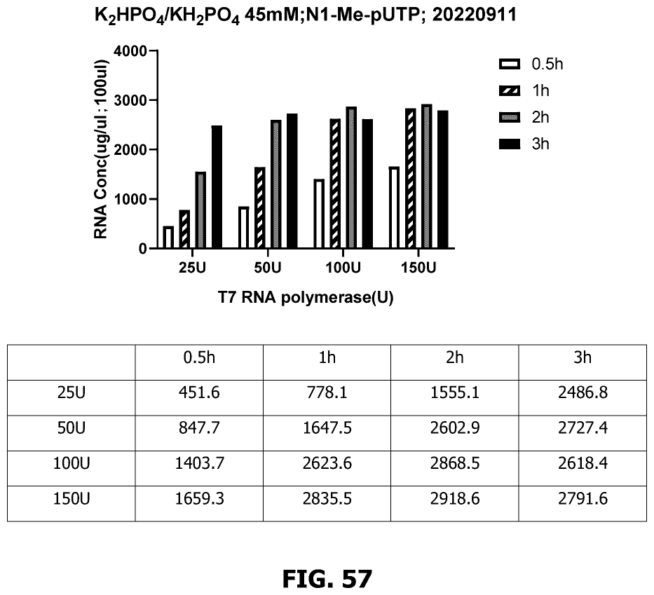

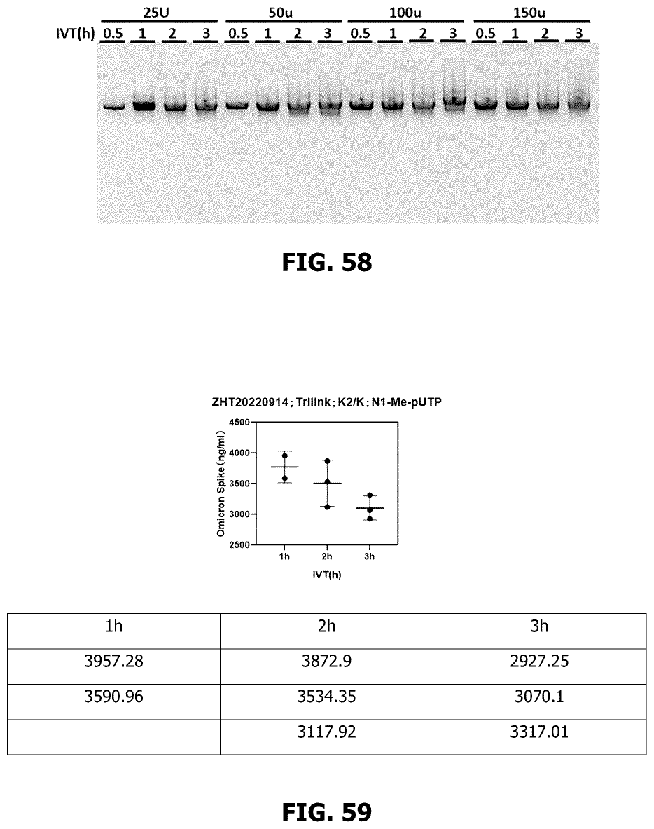

Experimental results are as shown in FIG. 57 to FIG. 59. The results indicate that a lower enzyme quantity needs a longer time to reach the upper limit of yield; however, longer reaction time is followed by lower purity, and the translation efficiency is decreased with the increase of time. Comprehensively, 100 U T7 reaction for 1 h is more conducive to the balance of yield, cost and translation efficiency.

5. Adaptability of K₂HPO₄/KH₂PO₄ in all in One

In view of the fact that phosphate is a novel transcription system, the present study hereby characterizes use in the All In One system. According to prior experience, adding DTT in the linearization stage is beneficial to linearization, and thus the present study adopted gradient addition of DTT in the linearization stage for detecting influence thereof on the phosphate system.

Components were formulated as shown in TABLE 39.

TABLE 39
Influence of Different Quantities og DTT Added on Different
Plasmid Linearization and Template Digestion

| | | |
|---|---|---|
| K₂HPO₄/KH₂PO₄ | 45 | mM |
| rATP(200 mM) | 14 | mM |
| rCTP(200 mM) | 14 | mM |
| rUTP(200 mM) | 14 | mM |
| rGTP(200 mM) | 14 | mM |
| CleanCap AG(100 mM) | 14 | mM |
| R53-S | 0.075 | μg/μL |
| MgAc₂(1M) | 50 | mM |
| CaCl₂(10 mM) | 0.5 | mM |
| Spermidine (100 mM) | 2.0 | mM |
| DTT (0.5M) | 0/6.25/12.5/25 | mM |

| | |
|---|---|
| RNase free Water | up to 20 μL |

All of the above used the template: R53 (SARS-CoV-2 Spike Omocron; 5951 bp) plasmid DNA, 0 mM (0 μL), 6.25 mM (0.25 μL), 12.5 mM (0.5 μL), and 25 mM (1 μL) DTT (500 mM) were added in sequence, mixed uniformly and placed at 50° C. to react for 1 h; at the end of reaction, 1 ul was pipetted into each tube to detect the linearization efficiency, and at the same time, IPP (100 U/mL) 0.15 U, DTT (500 mM) 25 mM, RNase Inhibitor (40,000 U/mL) 20 U and T7 polymerase (50,000 U/mL) 100 U were added in sequence, mixed uniformly and placed at 37° C. for 3 h; and at the end of reaction, 3 μL of DNase I was added, mixed uniformly, and reacted at 37° C. for 30/60 min. 1 μL was taken to detect template digestion, and the remaining was purified. Purification was performed by the lithium chloride precipitation method, and purity was detected by an agarose gel.

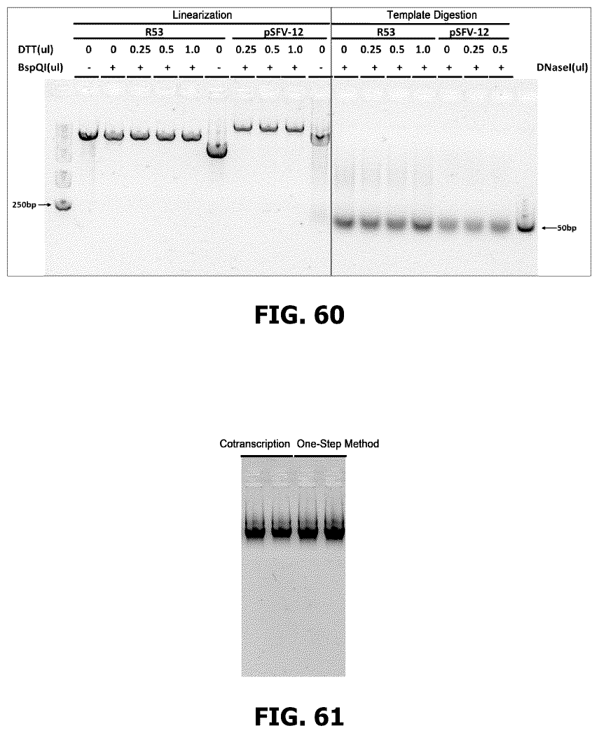

Experimental results are as shown in FIG. 60 and FIG. 61. The results indicate that adding a certain quantity of DTT in the linearization stage of the phosphate system helps to fully linearize the plasmid, and meanwhile, DNase I in the phosphate system can also degrade the template to a size of 50 bp, and finally the purity of mRNA prepared by the phosphate All In One is highly consistent with the conventional cotranscription process.

A linearized mRNA preparation system and use thereof and a preparation method for preparing mRNA by using the same provided in the present disclosure are introduced in detail in the above. The principle and embodiments of the present disclosure are described herein by way of specific examples, and the description of the above examples is only intended to help understand the method of the present disclosure and core idea thereof. It should be indicated that those skilled in the art still could make improvements and modifications to the present disclosure, without departing from the principle of the present disclosure, and all of these improvements and modifications all fall within the scope of protection of the claims of the present disclosure.

1. A linearization buffer system, comprising:

| | | |
|---|---|---|
| Tris-Ac or phosphate | 0-150 | mM; |
| divalent metal ions or trivalent metal ions | 0.5-150 | mM; |
| restriction endonuclease | 0.1-0.75 | U/μL; |
| spermidine | 0-7.5 | mM. |

2. The linearization buffer system according to claim 1, comprising:

| | | |
|---|---|---|
| Tris-Ac or phosphate | 0-120 | mM; |
| divalent metal ions or trivalent metal ions | 0.5-150 | mM; |
| restriction endonuclease | 0.1-0.75 | U/μL; |
| spermidine | 0-7.5 | mM. |

3. The linearization buffer system according to claim 1, wherein the phosphate has a concentration of 15-75 mM.

4. The linearization buffer system according to claim 1, wherein the phosphate has a pH value of 7.2-8.2.

5. The linearization buffer system according to claim 1, wherein the phosphate is added in a form of HPO₄²⁻/H₂PO₄⁻.

6. The linearization buffer system according to claim 1, wherein the HPO₄²⁻/H₂PO₄ is added in one or more forms of K₂HPO₄/KH₂PO₄, K₂HPO₄/NaH₂PO₄ or Na₂HPO₄/NaH₂PO₄.

7. The linearization buffer system according to claim 1, wherein the divalent metal ions comprise one or more of Mg²⁺, Ca²⁺, Mn²⁺ or Zn²⁺; or the trivalent metal ions comprise Fe³⁺.

8. The linearization buffer system according to claim 7, wherein the trivalent metal ions have a concentration of 25-150 mM; or
when the divalent metal ions are added in one or more forms of Mg²⁺, Mn²⁺ or Zn²⁺, the divalent metal ions have a concentration of 25-150 mM; or
when the divalent metal ions are added in the form of Ca²⁺, the divalent metal ions have a concentration of 0.5-1.5 mM.

9. The linearization buffer system according to claim 7, wherein the Ca²⁺ is added in a form of CaAc₂ or CaCl₂; or
the Mg²⁺ is added in a form of MgAc₂ or MgCl₂; or
the Mn²⁺ is added in a form of MnCl₂ or MnAc₂; or
the Zn²⁺ is added in a form of ZnSO₄; or
the Fe³⁺ is added in a form of FeCl₃.

10. (canceled)

11. A cotranscription system, comprising the linearization buffer system according to claim 1 and an acceptable enzyme or other adjuvants.

12. A preparation system of RNA, comprising the linearization buffer system according to claim 1 and an acceptable enzyme or other adjuvants.

13. The preparation system according to claim 12, further comprising: a transcription system, wherein
the transcription system comprises:

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM; |
| CAP | 2-18 | mM; |

or

| | | |
|---|---|---|
| T7 polymerase | 1.25-10 | U/μL; |
| IPP | 0-0.015 | U/μL; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM; |
| CAP | 2-18 | mM; |

or

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM. |

14. The preparation system according to claim 12, wherein the transcription system comprises:

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 7-16 | mM; |
| rCTP | 7-16 | mM; |
| rUTP | 7-16 | mM; |
| rGTP | 7-16 | mM; |
| CAP | 7-16 | mM; |

or

| | | |
|---|---|---|
| T7 polymerase | 1.25-10 | U/μL; |
| IPP | 0-0.015 | U/μL; |
| RNase inhibitor | 0-5 | U/μL; |
| rATP | 7-16 | mM; |
| rCTP | 7-16 | mM; |
| rUTP | 7-16 | mM; |
| rGTP | 7-16 | mM; |

or

| | | |
|---|---|---|
| T7 polymerase | 1.25-10 | U/μL; |
| IPP | 0-0.015 | U/μL; |
| DTT | 0-75 | mM; |
| RNase inhibitor | 0-5 | U/μL; |
| rATP | 2-18 | mM; |
| rCTP | 2-18 | mM; |
| rUTP | 2-18 | mM; |
| rGTP | 2-18 | mM. |

15. The preparation system according to claim 13, wherein the CAP is clean CAP AG.

16. The preparation system according to claim 12, wherein the preparation system comprises:

| | | |
|---|---|---|
| Tris-Ac | 40 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| restriction endonuclease | 0.2 | U/μL; |
| T7 polymerase | 5 | U/μL; |
| IPP | 0.00625 | U/μL; |
| DTT | 20 | mM; |
| RNase inhibitor | 1 | U/μL; |

or

| | | |
|---|---|---|
| Tris-Ac | 40 | mM; |
| rATP | 14 | mM; |
| rCTP | 14 | mM; |
| rUTP | 14 | mM; |
| rGTP | 14 | mM; |
| MgAc₂ | 50 | mM; |
| CaCl₂ | 0.5 | mM; |
| spermidine | 2.0 | mM; |
| restriction endonuclease | 0.2 | U/μL; |
| T7 polymerase | 5 | U/μL; |
| IPP | 0.00625 | U/μL; |
| DTT | 20 | mM; |
| RNase inhibitor | 1 | U/μL; |

or

| | | |
|---|---|---|
| Tris-Ac | 40 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| restriction endonuclease | 0.2 | U/μL; |
| T7 polymerase | 5 | U/μL; |
| IPP | 0.00625 | U/μL; |
| RNase inhibitor | 1 | U/μL; |

or

| | | |
|---|---|---|
| K₂HPO₄/KH₂PO₄ | 45 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| T7 polymerase | 5 | U/μL; |
| IPP | 0.0075 | U/μL; |
| DTT | 25 | mM; |
| RNase inhibitor | 1 | U/μL; |

or

| | | |
|---|---|---|
| K₂HPO₄/KH₂PO₄ | 30 | mM; |
| rATP | 14 | mM; |
| rCTP | 14 | mM; |
| rUTP | 14 | mM; |
| rGTP | 14 | mM; |
| Clean Cap AG | 14 | mM; |
| MgAc₂ | 50 | mM; |
| CaCl₂ | 0.5 | mM; |
| spermidine | 2.0 | mM; |
| T7 polymerase | 5 | U/μL; |
| IPP | 0.0075 | U/μL; |
| DTT | 25 | mM; |
| RNase inhibitor | 1 | U/μL. |

17. A preparation method for RNA, comprising: mixing the preparation system according to claim 12 with a template.

18. The preparation method according to claim 17, wherein the template has a concentration of 0-7.5 mM.

19. The preparation method according to claim 17, wherein the template has a concentration of 2 mM.

20. The preparation method according to claim 17, wherein the mixing comprises following steps:
S1: mixing the template with the linearization buffer system to render a sample; and
S2: mixing the sample obtained in S1 with the transcription system in a preparation system,
wherein the preparation system comprises a linearization buffer system and an acceptable enzyme or other adjuvants; and
the linearization buffer system comprises:

| | | |
|---|---|---|
| Tris-Ac or phosphate | 0-150 | mM; |
| divalent metal ions or trivalent metal ions | 0.5-150 | mM; |
| restriction endonuclease | 0.1-0.75 | U/μL; |
| spermidine | 0-7.5 | mM. |

21. The preparation method according to claim 17, after the mixing, further comprising: steps of removing DNA and centrifuging to remove supernatant.

22. (canceled)

23. (canceled)
